(19) United States
(12) Patent Application Publication
(10) Pub. No.: US 2008/0275219 A1
(43) Pub. Date: Nov. 6, 2008

(54) In Vitro Reconstitution of Ribonucleoprotein Complexes, and Methods of Use Therefor

(75) Inventors: Rachel Green, Baltimore, MD (US); Katharina Semrad, Vienna (AT)

Correspondence Address: EDWARDS ANGELL PALMER & DODGE LLP, P.O. BOX 55874, BOSTON, MA, 02205, US

(73) Assignee: The Johns Hopkins University, Baltimore, MD (US)

(21) Appl. No.: 11/630,595

(22) PCT Filed: Jun. 21, 2005
(86) PCT No.: PCT/US05/22017

§ 371 Date: Feb. 13, 2008

Related U.S. Application Data
(60) Provisional application No. 60/581,475, filed on Jun. 21, 2004.
(60) Provisional application No. 60/655,954, filed on Feb. 24, 2005.
(60) Provisional application No. 60/690,692, filed on Jun. 14, 2005.

(51) Int. Cl. C07K 14/195; C12N 9/10; C07H 21/00; C40B 40/10; C40B 40/08; C12P 21/02

(52) U.S. Cl. 530/358; 435/193; 536/24.33; 506/18; 506/17; 435/69.1

(57) ABSTRACT

In general, the invention provides methods and compositions for the in vitro production of ribonucleoprotein complexes and related multimolecular complexes useful for cell-free methods of protein production.

### REFERENCE TO RELATED APPLICATIONS

This application claims benefit of U.S. Provisional Application Ser. No. 60/581,475 filed on Jun. 21, 2004, U.S. Provisional Application Ser. No. 60/655,954 filed Feb. 24, 2005 and U.S. Provisional Application Serial No. Not Yet Assigned entitled “RNA CHAPERONE ACTIVITY OF LARGE RIBOSOMAL SUBUNIT PROTEINS FROM ESCHERICHIA COLI” filed Jun. 14, 2005 under Express Mail No. EV 492338711 US. The contents of each of these applications are incorporated herein by reference.

### GOVERNMENT SUPPORT

This work described herein was supported by a grant from the National Institute of Health (Grant No. GM59425). Therefore, the U.S. Government may have certain rights in the invention.

### BACKGROUND OF THE INVENTION

The in vitro synthesis of proteins is an important tool for molecular biologists and has a variety of applications, including proteomics, protein folding studies, and the incorporation of modified or unnatural amino acids for functional studies. The use of cell-free in vitro systems for translation offers a number of advantages over cellular gene expression, for example, when the over-expressed product is toxic to the host cell, when the gene product is insoluble or forms inclusion bodies, or when the protein undergoes rapid proteolytic degradation. Carrying out efficient in vitro protein synthesis also eliminates the need for costly and time-consuming cell culture. Currently, cell-free systems for protein synthesis rely on the use of lysates from rabbit reticulocytes, wheat germ, and Escherichia coli that contain macromolecular components required for translation. Such crude extracts are typically capable of generating only small quantities of proteins in vitro by incorporating natural amino acids into protein chains. A fully defined system capable of accomplishing the rapid and efficient synthesis of proteins and biopolymeric materials would offer significant advantages over existing technologies.

### SUMMARY OF THE INVENTION

The invention provides recombinant ribonucleoprotein complexes having the biological activity of native 50S ribosomal subunits. In addition, the invention provides recombinant multimolecular ribonucleoprotein complexes having protein and biopolymer synthesizing activity.

In one aspect, the invention generally features an isolated ribonucleoprotein complex containing a recombinant prokaryotic ribosomal polynucleotide and a recombinant prokaryotic ribosomal protein, or fragments thereof, where the ribonucleoprotein complex has a biological activity of a reference prokaryotic 50S ribosomal subunit.

In another aspect, the invention features an isolated ribonucleoprotein complex containing a recombinant polynucleotide having at least 85% nucleic acid sequence identity to a reference prokaryotic ribosomal polynucleotide sequence and a recombinant protein having at least 85% amino acid sequence identity to a reference prokaryotic ribosomal protein sequence, or fragments thereof, where the ribonucleoprotein complex has a biological activity of a naturally occurring prokaryotic 50S ribosomal subunit.

In another aspect, the invention features an isolated ribonucleoprotein complex containing a recombinant ribosomal prokaryotic polynucleotide of a bacteria and a recombinant ribosomal protein of a bacteria, or fragments thereof, where the ribonucleoprotein complex has a biological activity of a reference bacterial 50S ribosomal subunit.

In various embodiments of the above aspects, the recombinant protein and polynucleotide are at least 75%, 80%, 85%, 90%, 95%, or 97% identical to a ribosomal protein or polynucleotide of Escherichia coli, and the ribonucleoprotein complex has a biological activity of a naturally occurring Escherichia coli 50S ribosomal subunit.

In another aspect, the invention features an isolated ribonucleoprotein complex containing a recombinant ribosomal polynucleotide of Archaea and a recombinant ribosomal protein of Archaea, or fragments thereof, where the ribonucleoprotein complex has a biological activity of a reference 50S ribosomal subunit of Archaea.

In yet another aspect, the invention features an isolated ribonucleoprotein complex containing an in vitro transcribed prokaryotic 5S rRNA, an in vitro transcribed 23S rRNA, or fragments thereof, and at least one recombinant protein, selected from the group consisting of prokaryotic L1-L34 proteins, or fragments thereof, where the ribonucleoprotein complex has a biological activity of a reference prokaryotic 50S ribosomal subunit.

In a related aspect, the invention features an isolated ribonucleoprotein complex containing an in vitro transcribed 5S rRNA, an in vitro transcribed 23S rRNA, or fragments thereof, and more than one recombinant protein, or fragments thereof, selected from the group consisting of L1-L34 proteins, where the ribonucleoprotein complex has a biological activity of a reference 50S ribosomal subunit.

In yet another related aspect, the invention features an isolated ribonucleoprotein complex containing a recombinant prokaryotic 5S rRNA, a recombinant prokaryotic 23S rRNA, or fragments thereof, and prokaryotic ribosomal proteins L1-L34, or fragments thereof, where the ribonucleoprotein complex has a biological activity of a reference prokaryotic 50S ribosomal subunit.

In various embodiments of any of the above aspects, the 5S rRNA and the 23S rRNA have at least 75%, 85%, 90, 95%, or 97% nucleic acid sequence identity to a reference Escherichia coli rRNA and the ribosomal proteins L1-L34 have at least 75%, 85%, 90, 95%, or 97% amino acid sequence identity to a corresponding Escherichia coli ribosomal proteins. In various embodiments, the ribonucleoprotein complex comprises E. coli ribosomal proteins L1-L34.

In another aspect, the invention features a vector collection containing at least two vectors, each of the vectors containing a different polynucleotide encoding a ribosomal protein having at least 75%, 85%, 90, 95%, or 97% amino acid sequence identity to a prokaryotic ribosomal protein selected from the group consisting of L1-L34. In various embodiments of any of the above aspects, the vectors are expression vectors. In other embodiments, at least one of the polynucleotides is a degenerate polynucleotide sequence.

In yet another aspect, the invention features a primer collection, the collection containing at least 2 sets of primers, each set capable of binding to and amplifying a different polynucleotide encoding a prokaryotic ribosomal protein selected from the group consisting of L1-L34.

In yet another aspect, the invention features a cell collection containing at least two cells, where each of the cells comprises at least one vector of the vector collection of a previous aspect, where each of the vectors comprises a different polynucleotide. In one embodiment, each of the cells is present in a different cell line. In another embodiment, each of the cells is transiently transfected with the vector. In yet another embodiment, each of the cells expresses a prokaryotic ribosomal protein. In yet another embodiment, each of the cells expresses an E. coli protein selected from the group consisting of L1-L34 proteins.

In another aspect, the invention features a protein collection containing at least two different recombinant proteins, each of the proteins having at least 75%, 80%, 85%, 90%, 95%, or 97% amino acid sequence identity to a prokaryotic ribosomal protein selected from the group consisting of L1-L34. In one embodiment, each protein is a fusion protein. In another embodiment, each protein comprises an affinity tag. In yet another embodiment, each protein is affixed to a substrate. In yet another embodiment, the substrate is a bead, a membrane, a glass slide, a chip, or a filter.

In a related embodiment, the invention features a polynucleotide collection containing at least two different polynucleotides, each of the polynucleotides encoding a protein having at least 75%, 80%, 85%, 90%, 95%, or 97% amino acid sequence identity to a prokaryotic ribosomal protein selected from the group consisting of L1-L34. each of the polynucleotides is a prokaryotic ribosomal polynucleotide. In one embodiment, each polynucleotide comprises an affinity tag. In yet another embodiment, each polynucleotide is affixed to a substrate. In yet another embodiment, the substrate is a bead, a membrane, a glass slide, a chip, or a filter.

In another aspect, the invention features a microarray containing at least two nucleic acid molecules that encode a prokaryotic ribosomal protein selected from the group consisting of L1-L34, or fragments thereof.

In a related aspect, the invention features a microarray containing at least two prokaryotic ribosomal proteins, or fragments thereof, where the proteins are selected from the group consisting of L1-L34.

In another aspect, the invention features a method of producing a ribonucleoprotein complex, the method including the steps of: i. transcribing in vitro a prokaryotic 5S rRNA or 23S rRNA in the presence of at least one recombinant prokaryotic ribosomal protein under conditions that permit assembly of a ribonucleoprotein complex; and ii. isolating a ribonucleoprotein complex having the biological activity of a reference 50S ribosomal subunit.

In another aspect, the invention features a method of producing an isolated ribonucleoprotein complex, the method including the steps of: i. transcribing in vitro a prokaryotic 5S rRNA and a 23S rRNA in the presence of more than one recombinant prokaryotic ribosomal protein under conditions that permit assembly of a ribonucleoprotein complex; and ii. isolating a ribonucleoprotein complex having the biological activity of a reference 50S ribosomal subunit.

In another aspect, the invention features a method of producing an isolated ribonucleoprotein complex, the method including : i. transcribing in vitro a prokaryotic 5S rRNA and a 23S rRNA in the presence of recombinant prokaryotic ribosomal proteins L1-L34; and ii. isolating a ribonucleoprotein complex having the biological activity of a reference 50S ribosomal subunit.

A method of producing an isolated ribonucleoprotein complex, the method including: i. contacting recombinant prokaryotic 5S rRNA and 23S rRNA with at least one recombinant prokaryotic ribosomal protein selected from the group consisting of L1-L34; and ii. isolating a ribonucleoprotein complex the biological activity of a reference 50S ribosomal subunit.

In various embodiments of the above aspects, the recombinant prokaryotic 5S rRNA and 23S rRNA are contacted with prokaryotic ribosomal proteins L1-L34. In other embodiments, the ribonucleoprotein complex is a 50S ribonucleoprotein complex having the biological activity of an endogenous prokaryotic 50S ribonucleoprotein complex.

In another aspect, the invention features a method of producing an isolated ribonucleoprotein complex, the method including: i. transcribing in vitro a prokaryotic 5S rRNA and a 23S rRNA; ii. contacting at least one of the rRNAs with at least one recombinant ribosomal protein selected from the group consisting of L1-L34, iii. contacting the ribonucleoprotein complex of step ii with at least one additional ribosomal protein selected from the group consisting of L1-L34; iv. repeating steps ii-iii, until a ribonucleoprotein complex the biological activity of a reference 50S ribosomal subunit is formed; and v. isolating the ribonucleoprotein complex.

In another aspect, the invention features a method of producing an isolated 50S ribonucleoprotein complex, the method including: i. contacting a recombinant prokaryotic 5S rRNA and a recombinant prokaryotic 23S rRNA with at least one recombinant ribosomal protein selected from the group consisting of L1-L34; ii. contacting the ribonucleoprotein complex of step ii with at least one additional ribosomal protein; iii. repeating steps ii-iv to produce a 50S ribonucleoprotein complex containing ribosomal proteins L1-L34; and iv. isolating the 50S ribonucleoprotein complex.

In various embodiments of the above aspects, the method further includes the step of assaying the ribonucleoprotein complex for a biological activity of a reference 50S ribonucleoprotein complex. In related embodiments of the above aspects, the invention further includes the step of assaying the ribonucleoprotein complex for peptidyl transferase activity.

In yet other embodiments of the above aspects, the method further includes the step of assaying the ribonucleoprotein complex for a protein-protein or protein-RNA interaction that can be detected by mass spectroscopy. In still other embodiments of the above aspects, at least one of the ribosomal proteins or polynucleotides comprises an altered sequence relative to reference sequence. In still other embodiments of the above aspects, the method further comprises the step of optimizing the conditions under which the ribonucleoprotein complex is assembled by varying any one or more of the following conditions: i. the temperature; ii. salt concentration; iii. ribosomal protein concentration; iv. 5S rRNA concentration; v. 23S rRNA contration; vi. post-transcriptional modification of a 5S or 23S rRNA; vii. post-translational modification of a ribosomal protein; viii. presence of an additional agent that modulates the folding, association, or assembly of the ribonucleoprotein complex. In various embodiments, of the above aspects conditions are varied to produce a ribonucleoprotein complex having an altered rate of catalytic activity; having altered substrate specificity; having altered protein translation fidelity. In various embodiments of the above aspects, the 5S or 23S rRNA is incubated in an incubation buffer before or during ribonucleoprotein complex production. In other embodiments, the incubation buffer comprises a factor that enhances ribonucleoprotein complex production. In yet other embodiments, the factor is selected from the group consisting of an osmolyte, an antibiotic, a protein chaperone, and an RNA chaperone.

In another aspect, the invention features a ribonucleoprotein complex made by the process of any previous aspect, where the ribonucleoprotein complex has a biological activity of a reference 50S ribosomal subunit (e.g., peptidyl transferase activity, assayable by mass spectroscopy for protein or RNA composition).

In another aspect, the invention features an automated system for production of an isolated recombinant ribonucleoprotein complex, the system containing: i. a reaction substrate containing reaction reagents for the production of a ribonucleoprotein complex, where at least one of the reaction reagents is a recombinant 5S rRNA or a recombinant 23S rRNA; ii. a thermoelectric heating and cooling element on one side of the reaction substrate, where the element is configured to selectively heat or cool the reaction reagent; iii. a means for selectively depositing at least one reaction reagent on the substrate, where the depositing means is in communication with the reaction substrate; and iv. a means for isolating a recombinant ribonucleoprotein complex that contacts the reaction substrate. In one embodiment, the system further includes a means for detecting peptidyl transferase activity, where the means is in communication with the reaction components. In another embodiment, the system further further includes i. an electronic storage media in communication with the means for detecting peptidyl transferase activity; and ii. a display device that displays a representation of information relating to peptidyl transferase activity in communication with an electronic storage media.

In another aspect, the invention features a kit for ribonucleoprotein complex production, the kit containing i. an in vitro transcribed prokaryotic 5S rRNA; ii. an in vitro transcribed prokaryotic 23S rRNA; and iii. a plurality of recombinant ribosomal proteins selected from the group consisting of L1-L34.

In another aspect, the invention features a kit for ribonucleoprotein complex production, the kit containing i. a recombinant prokaryotic 5S rRNA; ii. a recombinant prokaryotic 23S rRNA; and iii. a plurality of recombinant ribosomal proteins selected from the group consisting of L1-L34.

In yet another aspect, the invention features a kit for ribonucleoprotein complex production, the kit containing i. reagents required for the in vitro transcription of a recombinant prokaryotic 5S rRNA; ii. reagents required for the in vitro transcription of a recombinant prokaryotic 23S rRNA; and iii. at least one recombinant ribosomal protein selected from the group consisting of L1-L34. In one embodiment, the kit further comprises an incubation buffer. In another embodiment, the incubation buffer comprises a factor that enhances ribonucleoprotein complex production (e.g., an osmolyte, an antibiotic, a protein chaperone, and an RNA chaperone).

In another aspect, the invention features a multimolecular complex containing: i. a 50S ribonucleoprotein complex containing an in vitro transcribed prokaryotic 5S rRNA, a 23S rRNA, and at least one prokaryotic recombinant ribosomal protein selected from the group consisting of L1-L34; ii. a 30S ribonucleoprotein complex containing an in vitro transcribed prokaryotic 16S rRNA and at least one prokaryotic recombinant ribosomal protein selected from the group consisting of S1-S21; where the multimolecular complex has a biological activity of a reference 70S ribosome.

In another aspect, the invention features a multimolecular complex containing: i. a 50S ribonucleoprotein complex containing a recombinant prokaryotic 5S rRNA, a recombinant 23S rRNA, and at least one recombinant prokaryotic ribosomal protein selected from the group consisting of L1-L34; and ii. a 30S ribonucleoprotein complex containing a recombinant prokaryotic 16S rRNA and at least one recombinant prokaryotic ribosomal protein selected from the group consisting of S1-S21; and where the multimolecular complex has a biological activity of a reference 70S ribosome.

In various embodiments of the above aspects, the complex comprises a recombinant prokaryotic polynucleotide of a bacteria and a recombinant protein of a bacteria, or fragments thereof, where the multimolecular complex has a biological activity of a reference 70S ribosome. In related embodiments, the biological activity is polymer synthesizing activity or protein synthesizing activity. In other embodiments of the above aspects, the 50S ribonucleoprotein complex has peptidyl tranferase activity. In still other embodiments of the above aspects, the 30S ribonucleoprotein complex has tRNA binding activity. In still other embodiments, the complex is associated with a nucleic acid template, or analog thereof, such as a template that includes a translation initiation region that includes an initiation codon or a Shine-Dalgarno (SD) sequence. In still other embodiments of the above aspects, the complex specifically accepts natural amino acid substrates for polymer synthesis. In yet other embodiments, the 50S or 30S ribonucleoprotein complex comprises an alteration in a polynucleotide or protein sequence relative to a reference sequence. In various embodiments, the alteration decreases substrate specificity; alters the rate of protein translation; alters protein translation fidelity; alters the association between the 30S and the 50S ribosome subunit; alters binding at a ribosomal tRNA binding site; alters binding at a ribosomal mRNA binding site; alters binding at a ribosomal IF3 binding site; alters binding at a ribosomal EF-Tu ribosomal binding site; alters binding at a binding at a ribosomal EF-G ribosomal binding site; or alters binding at any ribosomal active site.

In yet another aspect, the invention features a method of producing a multimolecular complex containing contacting a 50S ribonucleoprotein complex containing an in vitro transcribed prokaryotic 5S rRNA, a 23S rRNA, and at least one prokaryotic recombinant ribosomal protein selected from the group consisting of L1-L34 with a 30S ribonucleoprotein complex containing an in vitro transcribed prokaryotic 16S rRNA and at least one prokaryotic recombinant ribosomal protein selected from the group consisting of S1-S21 in the presence of a template and isolating a multimolecular complex having a biological activity of a reference 70S ribosome. In one embodiment, the method further includes the step of assaying the multimolecular complex for a biological activity of a reference 70S ribosome; the step of assaying the multimolecular complex for protein synthesizing activity; or the step of assaying the multimolecular complex for biopolymer synthesizing activity.

In yet another aspect, the invention features a multimolecular complex made by the process of any previous aspect.

In various embodiments of any of the above aspects, the multimolecular complex comprises a prokaryotic ribosomal protein having at least 75%, 80%, 85%, 90%, or 95% amino acid sequence identity to an E. coli ribosomal protein. In related embodiments, the multimolecular complex comprises a protein selected from the group consisting of E. coli L1-L34 proteins. In yet other embodiments, the multimolecular complex comprises a prokaryotic ribosomal protein having at least 75%, 80%, 85%, 90%, or 95% amino acid sequence identity to a protein selected from the group consisting of E. coli L1-L34 proteins. In other embodiments, the multimolecular complex comprises a prokaryotic ribosomal RNA having at least 75%, 80%, 85%, 90%, or 95% nucleic acid sequence identity to an E. coli 5S or 23S RNA. In other embodiments, at least one of the ribosomal proteins or polynucleotides comprises an altered sequence relative to reference sequence. In still other embodiments, the method further comprises the step of optimizing the conditions under which the multimolecular complex is assembled by varying any one or more of the following conditions: i. the temperature; ii. salt concentration; iii. ribosomal protein concentration; iv. 5S rRNA concentration; v. 23S rRNA contration; vi. 50S subunit concentration; vii. 30S subunit; vi. post-transcriptional modification of an rRNA; vii. post-translational modification of a ribosomal protein; and viii. presence of an additional agent that modulates the folding, association, or assembly of the multimolecular complex. In one embodiment, the 30S or 50S subunit is incubated in an incubation buffer before or during multimolecular complex production. In another embodiment, the incubation buffer comprises a factor that enhances multimolecular complex production. In yet another embodiment, the factor is selected from the group consisting of an osmolyte, an antibiotic, a protein chaperone, and an RNA chaperone.

In another aspect, the invention features a method of producing a recombinant protein, the method including the steps of: i. contacting the multimolecular complex of any previous aspect with a polynucleotide template under conditions that permit binding of the template to a component of the complex; ii. contacting the complex/template conjugate with a transfer RNA (tRNA) linked to an amino acid under conditions that permit binding of the tRNA; iii. contacting the complex/template conjugate with an additional tRNA linked to an amino acid under conditions that permit peptide bond formation between the amino acids; iv. repeating steps ii and iii under conditions that permit elongation of a an amino acid polymer; v. terminating the elongation of step iv; and vi. isolating the recombinant protein.

In another aspect, the invention features a method of producing a biopolymer, the method including the steps of: i. contacting the multimolecular complex of claim 110 or 111 with a template under conditions that permit binding of the template to a component of the complex; ii. contacting the complex/template conjugate with a tRNA, or tRNA analog, linked to a polymeric subunit under conditions that permit binding of the tRNA or tRNA analog to the template; iii. contacting the complex/template conjugate with an additional tRNA, or tRNA analog, linked to a polymeric subunit under conditions that permit bond formation between the polymeric subunits; iv. repeating steps ii and iii under conditions that permit elongation of the biopolymer; v. terminating the elongation of step iv; and vi. isolating the biopolymer.

In various embodiments, the method further includes the step of optimizing the conditions under which protein synthesis is carried out by varying a condition selected from the group consisting of: i. the temperature; ii. salt concentration; iii. post-transcriptional modification of a 5S or 23S rRNA; iv. post-translational modification of a ribosomal protein; v. presence of an additional agent that modulates the folding or assembly of the protein. In one embodiment, the multimolecular complex is present in an incubation buffer containing a factor that enhances protein synthesis.

In another aspect, the invention features a method of producing a recombinant ribosomal protein, the method including the steps of: i. contacting the multimolecular complex of any previous aspect with a polynucleotide template under conditions that permit binding of the template to a component of the complex; ii. contacting the complex/template conjugate with a transfer RNA (tRNA) linked to an amino acid under conditions that permit binding of the tRNA; iii. contacting the complex/template conjugate with an additional tRNA linked to an amino acid under conditions that permit peptide bond formation between the amino acids; iv. repeating steps ii and iii under conditions that permit elongation of an amino acid polymer; v. terminating the elongation of step iv; and vi. isolating the recombinant ribosomal protein.

In another aspect, the invention features a kit for cell-free recombinant protein synthesis, the kit containing: i. a 50S ribonucleoprotein complex containing a recombinant prokaryotic 5S rRNA, a 23S rRNA, and at least one recombinant ribosomal protein selected from the group consisting of L1-L34; and ii. a 30S ribonucleoprotein complex containing a recombinant prokaryotic 16S rRNA and at least one recombinant ribosomal protein selected from the group consisting of S1-S21. In one embodiment, the 50S ribonucleoprotein complex has a biological activity of a reference 50S subunit (e.g., peptidyl tranferase activity) or the complex has a protein or RNA composition that can be detected by mass spectroscopy. In another embodiment, the 30S ribonucleoprotein complex has tRNA binding activity.

In another aspect, the invention features a kit for biopolymer synthesis, the kit containing i. a 50S ribonucleoprotein complex containing a recombinant prokaryotic 5S rRNA, a 23S rRNA, and at least one recombinant ribosomal protein selected from the group consisting of L1-L34; and ii. a 30S ribonucleoprotein complex containing a recombinant prokaryotic 16S rRNA and at least one recombinant ribosomal protein selected from the group consisting of S1-S21. In one embodiment, the kit further contains at least one incubation buffer. In another embodiment, the incubation buffer comprises a component that functions in in vivo protein synthesis. In various embodiments, the component is a protein that alters the rate, accuracy, or specificity of protein synthesis (e.g., an initiation factor, elongation factor, or protein having chaperone activity).

In another aspect, the invention features an automated system for production of multimolecular complex of any previous aspect, the system containing: i. a reaction substrate containing reaction reagents, where at least one reagent is a recombinant 5S rRNA or a recombinant 23S rRNA; ii. a thermoelectric heating and cooling element on one side of the reaction substrate, where the element is configured to selectively heat or cool the reaction reagent; iii. a means for selectively depositing at least one reaction reagent on the substrate, where the depositing means is in communication with the reaction substrate; and iv. a means for isolating a recombinant multimolecular complex that contacts the reaction substrate. In one embodiment, the invention further includes a means for detecting production of a functional multimolecular complex, where the means is in communication with the reaction components. In another embodiment, the means detects protein synthesis. In another embodiment, the system, further includes i. an electronic storage media in communication with the means for detecting protein synthesis; and ii. a display device that displays a representation of information relating to peptidyl transferase activity in communication with an electronic storage media.

In various embodiments of any of the above aspects, the prokaryote is a bacterium selected from the group consisting of Actinobacteria, Aquificae, Bacteroidetes/Chlorobi group, Chlamydiae/Verrucomicrobia group, Chloroflexi, Chrysiogenetes, Cyanobacteria, Deferribacteres, Deinococcus-Thermus, Dictyoglomi, Fibrobacteres/Acidobacteria group, Firmicutes, Fusobacteria, Gemmatimonadetes, Nitrospirae, Planctomycetes, Proteobacteria, Spirochaetes, Thermodesulfobacteria, Thermotogae and unclassified bacteria. In other embodiments of any of the above aspects, the bacterium is selected from the group consisting of Aerobacter, Aeromonas, Acinetobacter, Actinomyces israelli, Agrobacterium, Bacillus, Bacillus antracis, Bacteroides, Bartonella, Bordetella, Bortella, Borrelia, Brucella, Burkholderia, Calymmatobacterium, Campylobacter, Citrobacter, Clostridium, Clostridium perfringers, Clostridium tetani, Cornyebacterium, corynebacterium diphtheriae, corynebacterium sp., Enterobacter, Enterobacter aerogenes, Enterococcus, Erysipelothrix rhusiopathiae, Escherichia, Francisella, Fusobacterium nucleatum, Gardnerella, Haemophilus, Hafnia, Helicobacter, Klebsiella, Klebsiella pneumoniae, Legionella, Leptospira, Listeria, Morganella, Moraxella, Mycobacterium, Neisseria, Pasteurella, Pasturella multocida, Proteus, Providencia, Pseudomonas, Rickettsia, Salmonella, Serratia, Shigella, Staphylococcus, Stentorophomonas, Streptococcus, Streptobacillus moniliformis, Treponema, Treponema pallidium, Treponema pertenue, Xanthomonas, Vibrio, and Yersinia.

In other embodiments of any of the above aspects, the recombinant protein and recombinant (e.g., in vitro transcribed) polynucleotide are at least 75%, 80%, 85%, 90%, or 95% identical to a ribosomal protein or polynucleotide of Escherichia coli, and the ribonucleoprotein complex has a biological activity of a naturally occurring Escherichia coli 50S ribosomal subunit. In other embodiments of the above aspects, the recombinant protein and polynucleotide are at least 75%, 80%, 85%, 90%, or 95% identical to a ribosomal protein or polynucleotide of Escherichia coli, and the ribonucleoprotein complex has a biological activity of a naturally occurring Escherichia coli 50S ribosomal subunit.

In yet other embodiments of any of the above-aspects, the invention features an isolated ribonucleoprotein complex containing a recombinant ribosomal polynucleotide of Archaea and a recombinant ribosomal protein of Archaea, or fragments thereof, where the ribonucleoprotein complex has a biological activity of a reference 50S ribosomal subunit of Archaea. In various embodiments of any of the above aspects, the prokaryote is an archaea selected from the group consisting of Crenarchaeota, Euryarchaeota, Korarchaeota, Nanoarchaeota and unclassified Archaea. In other embodiments, the Crenarchaeota is a Thermoprotei; the Euryarchaeota is selected from the group consisting of Archaeoglobi, Halobacteria, Methanobacteria, Methanococci, Methanonmicrobia, Methanopyri, Thermococci, and Thermoplasmata; or the Archaea is selected from the group consisting of Aeropyrum pernix, Archaeoglobus fulgidus, Halobacterium sp., Methanococcus jannaschii, Methanobacterium thermoautotrophicum, Pyrococcus abyssi, Pyrococcus horikoshii, and Thermoplasma acidophilum.

In yet other embodiments of the above aspects, the biological activity is peptidyl transferase activity. In other embodiments of any of the above aspects, the composition of a complex (e.g., ribonucleoprotein or multimolecular) can be assayed using mass spectroscopy. In one embodiment of that aspect, mass spectroscopy assay a protein-protein or protein-RNA interaction.

In yet other embodiments of any of the above aspects, the prokaryote is a mesophile, a thermophile, a moderate thermophile, an extreme thermophile, a hyperthermophile, or a psychrophile. In still other embodiments of any of the above aspects, the recombinant polynucleotide is a 5S ribosomal RNA (rRNA), or fragment thereof; the recombinant polynucleotide is a 23S rRNA, or fragment thereof; or the recombinant polynucleotide is a 16S rRNA, or fragment thereof. In still other embodiments of the above aspects, the ribosomal proteins are selected from the group consisting of E. coli L1-L34. In various embodiments of any of the above aspects, the encoded ribosomal proteins comprise affinity tags or detectable amino acid sequences.

In still other embodiments of any of the above aspects, the complex contains a prokaryotic ribosomal protein having at least 75%, 80%, 85%, 90%, or 95% amino acid sequence identity to a protein selected from the group consisting of E. coli L1-L34 proteins. In yet other embodiments, the ribonucleoprotein complex contains a prokaryotic ribosomal RNA having at least 75%, 80%, 85%, 90%, or 95% nucleic acid sequence identity to a an E. coli 5S or 23S RNA. In other embodiments of any of the above aspects, the ribonucleoprotein complex consists essentially of a prokaryotic 5S rRNA, a 23S rRNA, and ribosomal proteins L1-L34.

### BRIEF DESCRIPTION OF THE DRAWINGS

FIGS. 1A and 1B show stimulation of trans splicing by ribosomal protein L19. FIG. 1A is a schematic diagram showing the trans splicing assay: RNAs H1 and H2 are annealed and splicing is started by the addition of the guanosine cofactor. FIG. 1B shows a trans splicing polyacrylamide (PAA) gel. The substrates H1, H2 and the product (guanosine-5′-intron) are indicated. The first set (lanes 1-6) shows a time course of trans splicing in the absence of proteins at 55° C. and the second set (lanes 7-12) show splicing at 37° C. The third set (lanes 13-18) show trans splicing at 37° C. in the presence of 2 μM L19. The final set (lanes 19-24) show trans splicing in the presence of ribosomal protein storage buffer.

FIG. 2 is a polyacrylamide gel that shows a time course of trans splicing in the absence of ribosomal proteins at 55° C. (first set of lanes 1-4), and at 37° C. (second set of lanes 5-8). It follows the time courses in the presence of 2 μM ribosomal proteins L1, L9, L7/L12, L13 and L15. Lanes indicated with “b” correspond to sets of trans splicing performed in the presence of ribosomal protein storage buffer, where the same volume as for each ribosomal protein was used.

FIG. 3 is a graph that shows trans splicing rates in the absence and in the presence of ribosomal proteins L1 to L34 from E. coli. The first two bars, which are labeled 55° and 37°, show relative trans splicing rates at 55° C. and 37° C. in the absence of proteins. The following bars show relative rates of trans splicing in the presence of the indicated ribosomal protein at 2 μM final concentration. L1, L3, L13, L15, L16, L18, L19, L22, and L24 show strong RNA chaperone activities. Ribosomal proteins L4 and L17 show intermediate stimulation of trans splicing. Relative splicing rates are obtained from 1 to 5 individual experiments and calculated by the formula (nx-n37)/(n55-n37) for each PAA gel separately. “n” is the relative splicing rate of either “x” (the respective ribosomal protein) or “55” (at 55° C.) or “37” (at 37° C., the latter two in the absence of a protein).

FIG. 4 is a schematic diagram showing the steps typically used for 50S in vitro reconstitution.

FIG. 5 shows the sequences of polynucleotides encoding E. coli ribosomal proteins L1-L34.

### DETAILED DESCRIPTION OF THE INVENTION

### Definitions

By “ribonucleoprotein complex” is meant an assembly of at least one ribosomal RNA specifically bound to at least one ribosomal protein, where the RNA and protein are the product of genetic engineering; and where the RNA/protein complex has a biological activity associated with a naturally occurring ribonucleoprotein complex (e.g., a ribonucleoprotein complex derived from a 30S or 50S subunit).

By “ribosomal protein” is meant a polypeptide, or fragment thereof, that in its naturally occurring state is contained within or associated with a ribosome (e.g., a prokaryotic ribosome). Exemplary ribosomal proteins include, but are not limited to, polypeptides that form a prokaryotic 50S or 30S ribosomal subunit, such as ribosomal proteins L1-L34 or S1-S21, and orthologs or variants thereof.

By “ribosomal polynucleotide” is meant a nucleic acid molecule, or fragment thereof, that in its natural state is found in a ribosome (e.g., a ribosomal RNA) or that encodes a ribosomal protein.

By “ribosomal L1-L34 protein” is meant a polypeptide or fragment thereof that forms part of a prokaryotic 50S ribosomal subunit. Exemplary L1-L34 proteins are those derived from an E. coli 50S subunit as well as their homologs. In other embodiments a ribosomal L1-L34 protein has at least 50%, 75%, 85%, 90%, or 95% amino acid identity to an E. Coli L1-L34 protein and is naturally found in a prokaryotic ribosome. Other L1-L34 ribosomal proteins within the scope of the invention are orthologs of an L1-L34 protein. The amino acid sequences of E. coli ribosomal L1-L34 proteins are encoded by the following EMBL Accession Nos:

L1: V00339, (604-1308); L2: U18997, (232112-231291); L3: U18997, (233674-233045); L4: U18997, (233034-232429); L5: U18997, (228186-227647); L6: U18997, (226888-226355); L9: X04022, (1439-1888); L10:V00339, (1721-2218); L11: V00339, (172-600); L7/L12: V00339, (2285-2650); L13: U18997, (159396-158968); L14: U18997, (228897-228526); L15: U18997, (225287-224853); L16: U18997, (229917-229507); L17: U18997, (220747-220364); L18: U18997, (226345-225992); L19: X01818, (3426-3773); L20: AE000266, (1797417-1797773); L21: U18997, (114203-113892); L22: U18997, (230981-230649); L23: U18997, (232432-232130); L24: U18997, (228515-228201); L25: D13326, (382-666): L27: U18997, (113871-113614); L28: L10328, (1066-830); L29: U18997, (229507-229316); L30: U18997, (225470-225291); L31:L19201, (88646-88858); L32: M29698, (868-1041); L33: L10328, (809-642); L34: L10328, (73734-73874).

By “ribosomal L1-L34 polynucleotides” are meant nucleic acid molecules that encode a ribosomal L1-L34 protein, respectively.

By “biological activity of a 50S ribosomal subunit” is meant any function associated with a naturally occurring 50S ribosomal subunit. Accordingly, biological activities of a 50S ribosomal subunit include peptidyl transferase activity, association with a 30S subunit, or binding of amino acyl tRNA.

By “biological activity of a 30S ribosomal subunit” is meant any function associated with a naturally occurring 30S ribosomal subunit. Accordingly, biological activities of a 30S ribosomal subunit include tRNA binding, mRNA binding, and association with a 50S subunit.

By “multimolecular complex” is meant an assembly containing at least 2 ribonucleoprotein complexes and having a biological activity associated with a naturally occurring ribosome or ribosomal subunit (e.g., a 30S or 50S subunit). The multimolecular complex may optionally contain a template with which it is associated or may be assembled in the absence of a template.

By “biological activity of a multimolecular complex” or “biological activity of a 70S particle” is meant any function associated with a naturally occurring prokaryotic ribosome. Accordingly, biological activities of a multimolecular complex include any function of a 30S or 50S subunit, as well as protein or biopolymer synthesizing activity.

By “template” is meant a nucleic acid molecule or analog thereof that encodes the synthesis of a protein or biopolymer.

By “affinity tag” is meant any moiety used for the purification of a protein, polynucleotide, or ribonucleoprotein complex to which it is fixed.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, for example, hydroxyproline, gamma-carboxyglutamate, and O-phosphoserine, phosphothreonine.

By an “amino acid analog” is meant a compound that has the same basic chemical structure as a naturally occurring amino acid, i.e., a carbon that is bound to a hydrogen, a carboxyl group, an amino group, and an R group (e.g., homoserine, norleucine, methionine sulfoxide, methionine methyl sulfonium), but that contain some alteration not found in a naturally occurring amino acid (e.g., a modified side chain); the term “amino acid mimetic” refers to chemical compounds that have a structure that is different from the general chemical structure of an amino acid, but that function in a manner similar to a naturally occurring amino acid. Amino acid analogs may have modified R groups (for example, norleucine) or modified peptide backbones, but retain the same basic chemical structure as a naturally occurring amino acid. In one embodiment, an amino acid analog is a D-amino acid, a β-amino acid, or an N-methyl amino acid.

Amino acids and analogs are well known in the art. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

By “cell collection” is meant at least two cells each of which contains a different recombinant polynucleotide. In one embodiment, the cell is a bacterial cell transformed with a plasmid containing that encodes a ribosomal polynucleotide. In another embodiment, a mammalian cell is transiently transfected or stably transfected with an L1-L34 encoding polynucleotide, such that the cells are clonal members of a cell line. By “collection” is meant a group having at least 2, 5, 10, 15, 20, 25, 30, 35, 40, 45, or 50 members.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

By “detectable amino acid sequence” or “detectable moiety” is meant a composition that when linked with the nucleic acid or protein molecule of interest renders the latter detectable, via any means, including spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

A “labeled nucleic acid or oligonucleotide probe” is one that is bound, either covalently, through a linker or a chemical bond, or noncovalently, through ionic bonds, van der Waals forces, electrostatic attractions, hydrophobic interactions, or hydrogen bonds, to a label such that the presence of the nucleic acid or probe may be detected by detecting the presence of the label bound to the nucleic acid or probe.

By “fragment” is meant a portion (e.g., at least 10, 25, 50, 100, 125, 150, 200, 250, 300, 350, 400, or 500 amino acids or nucleic acids) of a protein or nucleic acid molecule that is substantially identical to a reference protein or nucleic acid and retains the biological activity of the reference. In some embodiments the portion retains at least 50%, 75%, or 80%, or more preferably 90%, 95%, or even 99% of the biological activity of the reference protein or nucleic acid described herein.

A “host cell” is any prokaryotic or eukaryotic cell that contains either a cloning vector or an expression vector. This term also includes those prokaryotic or eukaryotic cells that have been genetically engineered to contain the cloned gene(s) in the chromosome or genome of the host cell.

The terms “isolated,” “purified,” or “biologically pure” refer to material that is free to varying degrees from components which normally accompany it as found in its native state. Various levels of purity may be applied as needed according to this invention in the different methodologies set forth herein; the customary purity standards known in the art may be used if no standard is otherwise specified.

By “isolated nucleic acid molecule” is meant a nucleic acid (e.g., a DNA, RNA, or analog thereof) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule which is transcribed from a DNA molecule, as well as a recombinant DNA which is part of a hybrid gene encoding additional polypeptide sequence.

“Microarray” means a collection of nucleic acid molecules or polypeptides from one or more organisms arranged on a solid support (for example, a chip, plate, or bead).

By “nucleic acid” is meant an oligomer or polymer of ribonucleic acid or deoxyribonucleic acid, or analog thereof. This term includes oligomers consisting of naturally occurring bases, sugars, and intersugar (backbone) linkages as well as oligomers having non-naturally occurring portions which function similarly. Such modified or substituted oligonucleotides are often preferred over native forms because of properties such as, for example, enhanced association with a ribonucleoprotein or multimolecular complex, and increased stability in the presence of nucleases. In one embodiment, a modified nucleic acid molecule is found at a ribosomal active site, such that the modified nucleic acid molecule imparts a desirable alteration in the active site (e.g., altered biological activity).

Specific examples of some nucleic acids envisioned for this invention may contain phosphorothioates, phosphotriesters, methyl phosphonates, short chain alkyl or cycloalkyl intersugar linkages or short chain heteroatomic or heterocyclic intersugar linkages. Also preferred are oligonucleotides having morpholino backbone structures (Summerton, J. E. and Weller, D. D., U.S. Pat. No: 5,034,506). In other preferred embodiments, such as the protein-nucleic acid (PNA) backbone, the phosphodiester backbone of the oligonucleotide may be replaced with a polyamide backbone, the bases being bound directly or indirectly to the aza nitrogen atoms of the polyamide backbone (P. E. Nielsen et al. Science 199: 254, 1997). Other preferred oligonucleotides may contain alkyl and halogen-substituted sugar moieties comprising one of the following at the 2′ position: OH, SH, SCH₃, F, OCN, O(CH₂)ₙNH₂ or O(CH₂)ₙ CH₃, where n is from 1 to about 10; C₁ to C₁₀ lower alkyl, substituted lower alkyl, alkaryl or aralkyl; Cl; Br; CN; CF₃; OCF₃; O—, S—, or N-alkyl; O—, S—, or N-alkenyl; SOCH₃; SO₂CH₃; ONO₂; NO₂; N₃; NH₂; heterocycloalkyl; heterocycloalkaryl; aminoalkylamino; polyalkylamino; substituted silyl; an RNA cleaving group; a conjugate; a reporter group; an intercalator; a group for improving the pharmacokinetic properties of an oligonucleotide; or a group for improving the pharmacodynamic properties of an oligonucleotide and other substituents having similar properties. Oligonucleotides may also have sugar mimetics such as cyclobutyls in place of the pentofuranosyl group. Other preferred embodiments may include at least one modified base form. Some specific examples of such modified bases include 2-(amino)adenine, 2-(methylamino)adenine, 2-(imidazolylalkyl)adenine, 2-(aminoalklyamino)adenine, or other heterosubstituted alkyladenines. By “operably linked” is meant that a first polynucleotide is positioned adjacent to a second polynucleotide that directs transcription of the first polynucleotide when appropriate molecules (e.g., transcriptional activator proteins) are bound to the second polynucleotide.

By “ortholog” is meant any protein or nucleic acid molecule of an organism that is highly related to a reference protein or nucleic acid sequence from another organism.

By “recombinant” is meant the product of genetic engineering or chemical synthesis.

By “protein” is meant any chain of amino acids, or analogs thereof, regardless of length or post-translational modification.

By “positioned for expression” is meant that the polynucleotide of the invention (e.g., a DNA molecule) is positioned adjacent to a DNA sequence that directs transcription and translation of the sequence (i.e., facilitates the production of, for example, a recombinant protein of the invention, or an RNA molecule).

By “primer set” is meant a pair of oligonucleotides that may be used, for example, for PCR.

By “primer collection” is meant at least 2 primer sets. In general a primer collection contains at least 2, 4, 6, 8, 10, 12, 14, 16, 18, 20, 30, 40, 50, 60, 68, 80, 100, 200, 250, 300, 400, 500, 600, or more primers.

An “expression vector” is a nucleic acid construct, generated recombinantly or synthetically, bearing a series of specified nucleic acid elements that enable transcription of a particular gene in a host cell. Typically, gene expression is placed under the control of certain regulatory elements, including constitutive or inducible promoters, tissue-preferred regulatory elements, and enhancers.

By “reference” is meant a standard or control condition.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For proteins, the length of the reference protein sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

By “specifically binds” is meant a compound or antibody that recognizes and binds a protein of the invention, but which does not substantially recognize and bind other molecules in a sample, for example, a biological sample, which naturally includes a protein of the invention.

By “substantially identical” is meant a protein or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and most preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “transformed cell” is meant a cell into which (or into an ancestor of which) has been introduced, by means of recombinant DNA techniques, a polynucleotide molecule encoding (as used herein) a protein of the invention.

### Methods of the Invention

The invention provides recombinant ribonucleoprotein complexes having the biological activity of native 50S ribosomal subunits. In addition, the invention provides recombinant multimolecular ribonucleoprotein complexes containing recombinant proteins and in vitro synthesized ribosomal RNAs. These multimolecular complexes have protein and biopolymer synthesizing activity. The invention also provides methods of using these complexes for in vitro protein and biopolymer synthesis. Accordingly, the invention provides a number of advantages over existing methods of cell-free protein synthesis. Because the system is fully defined, protein synthesis is carried out in the absence of proteases and nucleases, allowing the generation of increased quantities of protein. Purification of the synthesized proteins is facilitated by the use affinity tagged multimolecular complex components, which reduces problems associated with protein purification. Moreover, the present invention provides for incorporation of nonnatural building blocks, thereby expanding the genetic code.

### Reconstituted 50S Subunits

In vitro transcribed prokaryotic 5S and 23S ribosomal RNAs and recombinant prokaryotic ribosomal proteins L1-L34 may be used to reconstitute a 50S ribosomal subunit. In general, a ribonucleoprotein complex having the biological activity of a 50S subunit is obtained by overexpressing and purifying large quantities of each of the Escherichia coli ribosomal proteins L1-L34 from plasmid-borne copies of the ribosomal protein genes. The expressed proteins are then purified using standard methods. The expressed proteins are then combined with in vitro transcribed 5S and 23S ribosomal RNAs (rRNAs). Methods for the reconstitution of functional 50S transcripts from in vitro transcripts or chemically synthesized transcripts and isolated proteins are known in the art. (See Semrad and Green RNA 8:401-411, 2002). Such methods are also useful for the reconstitution of 50S ribosomal subunits from in vitro transcripts and recombinant proteins.

In vitro reconstitution of E. coli 50S subunits

Methods for the in vitro reconstitution of 50S subunits are known in the art and were first described by Dohme and Nierhaus in 1976. Current methods for in vitro reconstitution of 50S subunits are described, for example, by Green and Noller (Biochemistry. Feb. 9, 1999;38(6):1772-9) and by Khaitovich et al., (Biochemistry. Feb. 9, 1999;38(6):1780-8). In one embodiment, in vitro-transcribed 23S rRNA and in vitro transcribed 5S rRNA is incubated with recombinant TP50 (total proteins that are naturally present in a prokaryotic 50S subunit) at low temperature (e.g., 40° C.) in low magnesium (4 mM), which is then followed by incubation at higher temperature (e.g., 50° C.) in high magnesium (20 mM).

In one embodiment, reconstitution is carried out in a suitable buffer (e.g., 20 mM Tris-HCl, 7+4, 4 mM MgAc, 400 mM NH4Cl, 0+2 mM EDTA, and 5 mM β-mercaptoethanol). The reaction mixture is incubated in this buffer for 20 minutes at 44° C. Then the magnesium concentration is increased to 20 mM and the reaction is incubated at 50° C. for 90 minutes. Reconstitution may be carried out in the presence of any reagent (e.g., osmolytes, antibiotics, RNA chaperones, chemical chaperones, denaturants, and purified components of cellular extracts (e.g., S100 extracts) known or identified as enhancing the in vitro assembly of a functional 50S subunit or of a 50S subunit having a desired characteristic (e.g., acceptance of altered substrates, altered translational accuracy) from in vitro transcribed RNAs and recombinant ribosomal proteins. 50S subunits may also be reconstituted using, for example, ordered assembly or transcriptional assembly.

Ordered Assembly

An ordered assembly protocol is described by Culver and Noller (Methods in Enzymology 318: 446-460, 2000). This protocol describes the efficient in vitro reconstitution of a functional 30S subunit. Similar methods are used to assemble a functional 50S subunit from in vitro transcribed rRNAs and recombinant ribosomal proteins. Such methods are described, for example, by Rohl and Nierhaus (Proc Natl Acad Sci USA. February 1982;79(3):729-33).

The ordered assembly procedure is based on the addition of recombinant proteins in ordered protein mixtures, where the order of addition is determined empirically. In one embodiment, the ratio of protein added is determined by titrating the ratio of protein to RNA, while monitoring the efficiency of reconstitution by sucrose gradient sedimentation to identify ribonucleoprotein complexes having a sedimentation value of 70S. Recombinant ribonucleoprotein complexes are then assayed for synthetic activity or using mass spectrometry to identify the protein and RNA composition of the 50S subunit.

Co-Transcriptional Assembly

In contrast to ordered assembly, which relies on the sequential addition of individual proteins or protein mixtures, transcriptional assembly provides for the in vitro transcription of the components of a 50S ribonucleoprotein complex in the presence of a mixture of recombinant ribosomal proteins.

### Compounds that Enhance Reconstitution

Two different classes of compound have been shown to increase reconstitution efficiency of reconstituted ribosomes made from in vitro-transcribed 23S rRNA. In one embodiment, the ketolide antibiotic telithromycin (HMR3647) is added to a 50S reconstitution reaction. Its effect on a recombinant 50S subunit is then assayed using the fragment peptidyl transferase assay. A second class of compounds known as osmolytes also enhance the activity of a 50S reconstitution reaction. Two particular compounds, trimethylamine-oxide (TMAO) and betaine (both containing a trimethylamine moiety), are likely to be most effective in stimulating the reconstitution reaction as measured in the fragment reaction.

Combinations of telithromycin and the osmolyte TMAO are also likely to be useful in enhancing the reconstitution efficiency of recombinant 50S subunits. The activity of various combinations of reagents, is assessed using the fragment reaction to assay the combined activity of telithromycin and TMAO on the reconstitution efficiency. Another useful reagent expected to enhance the activity of a reconstituted 50S subunit is the RNA chaperone StpA. Combinations of these and other reagents are also assessed using the intact tRNA peptidyl transferase assay.

Osmolytes useful in the methods of the invention are known in the art, and are described, for example, by Semrad et al., RNA 8:401-411, 2002. Osmolytes useful in the methods of the invention include, but are not limited to, telithromycin, trimethylamine oxide (TMAO) and betaine. Desirably, the 50S subunit generated by such methods is used in the assembly of a multimolecular complex.

### Methods for Identifying Reagents that Enhance Reconstitution

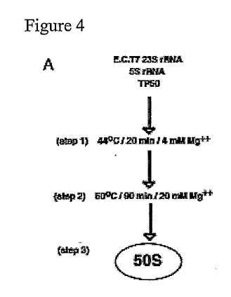

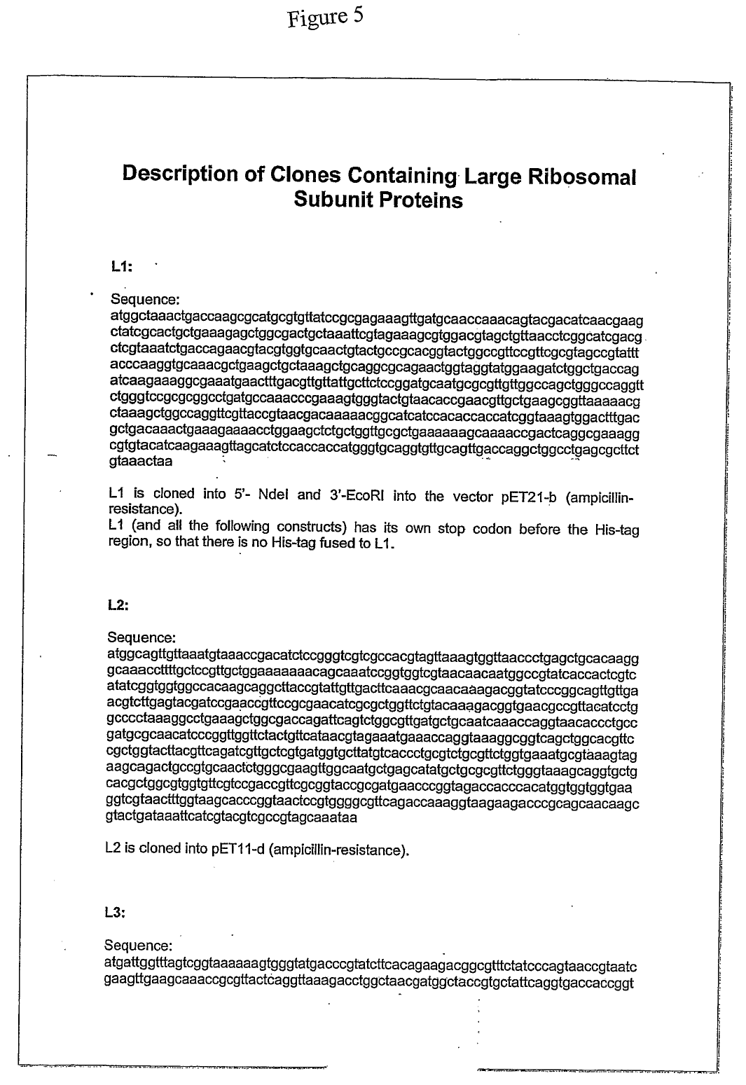

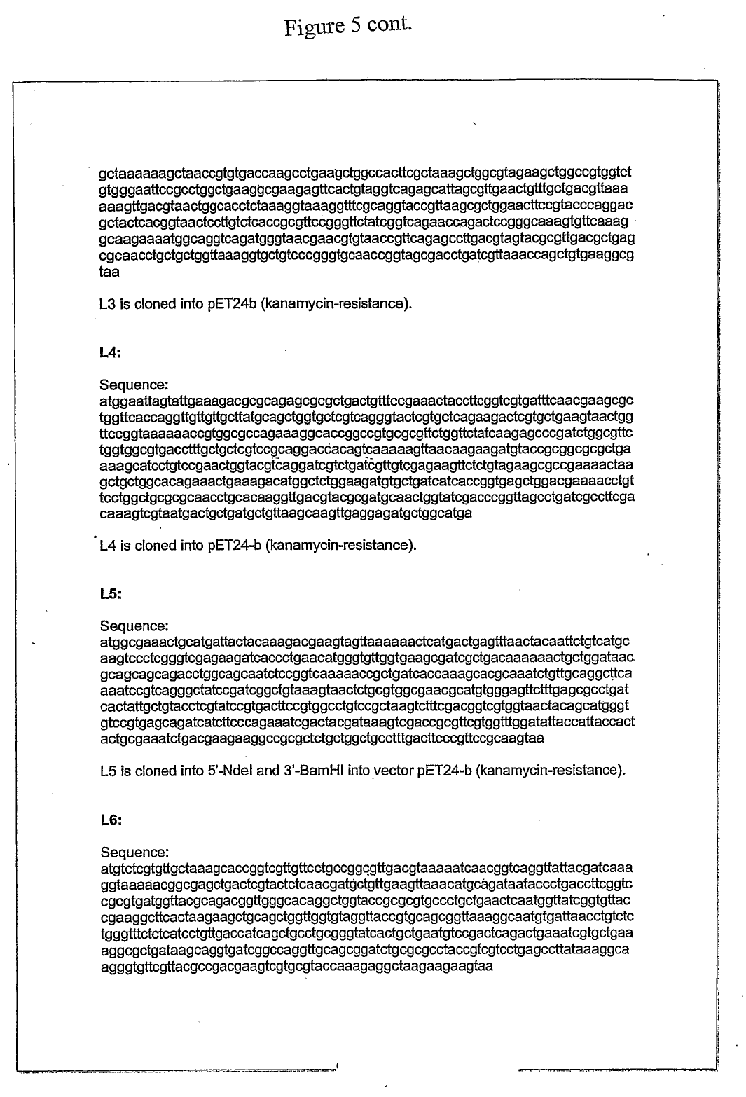

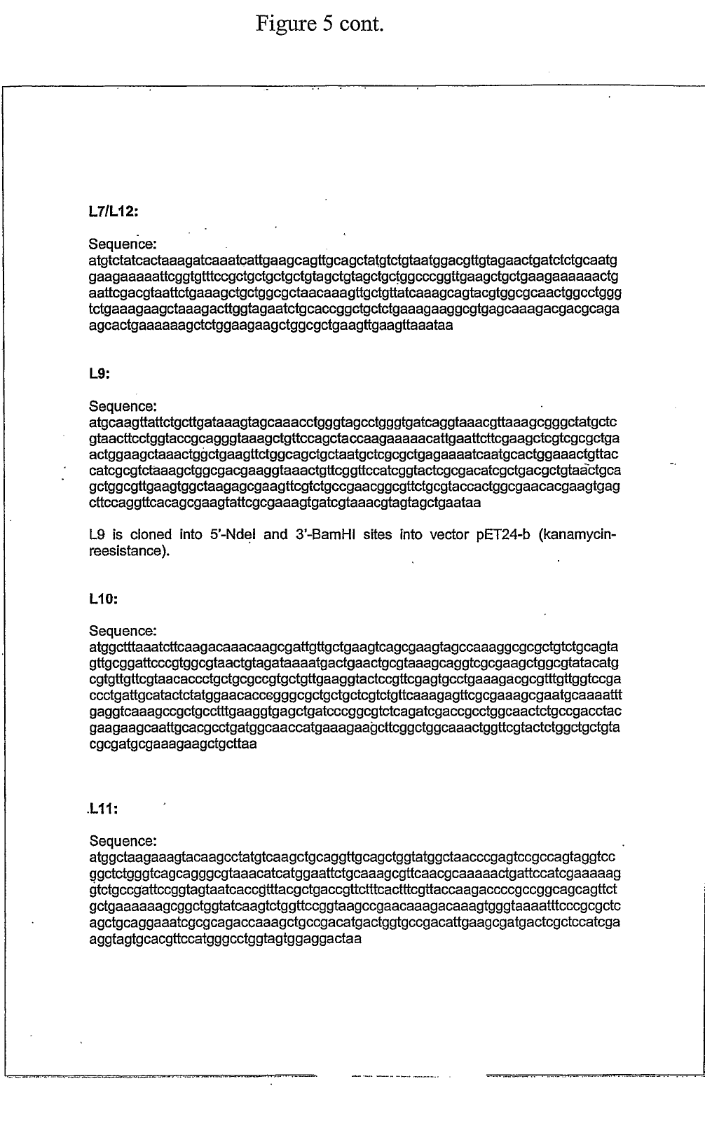

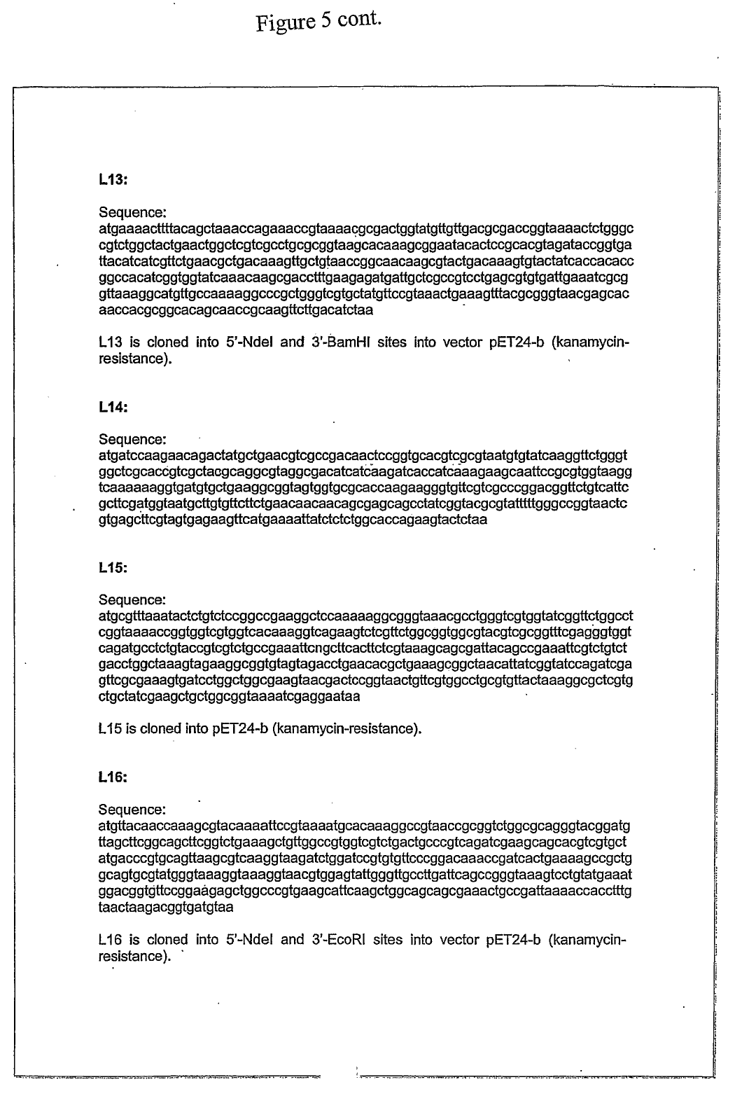

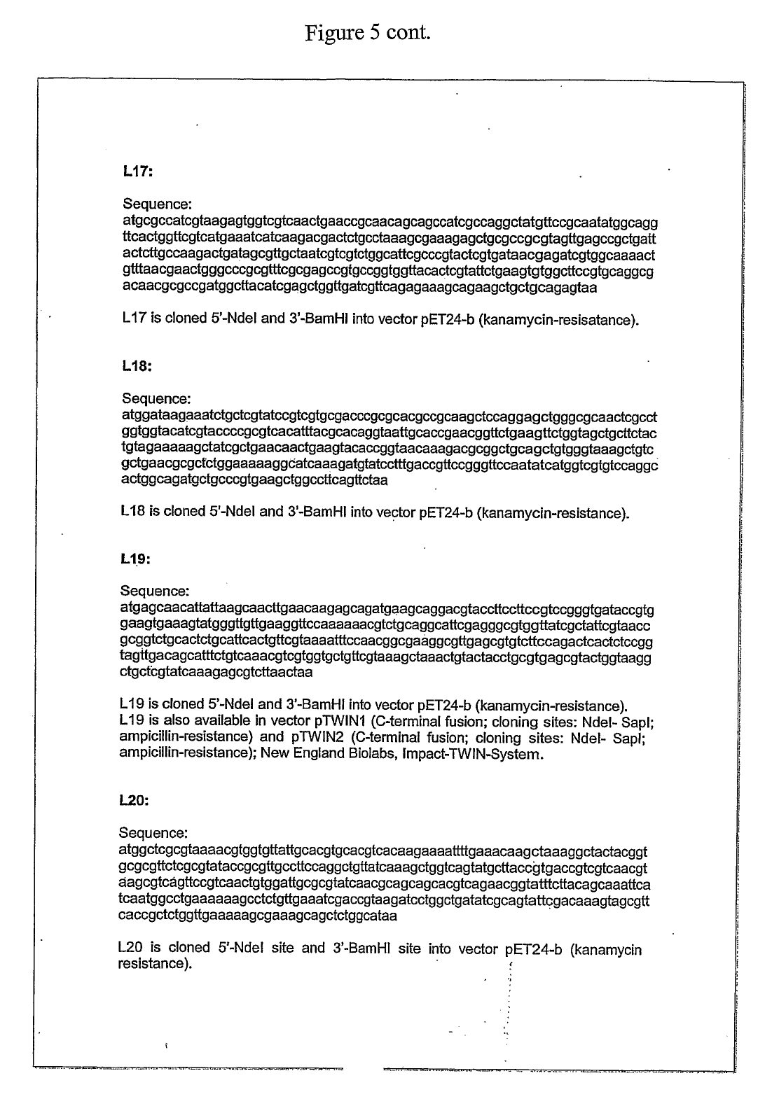

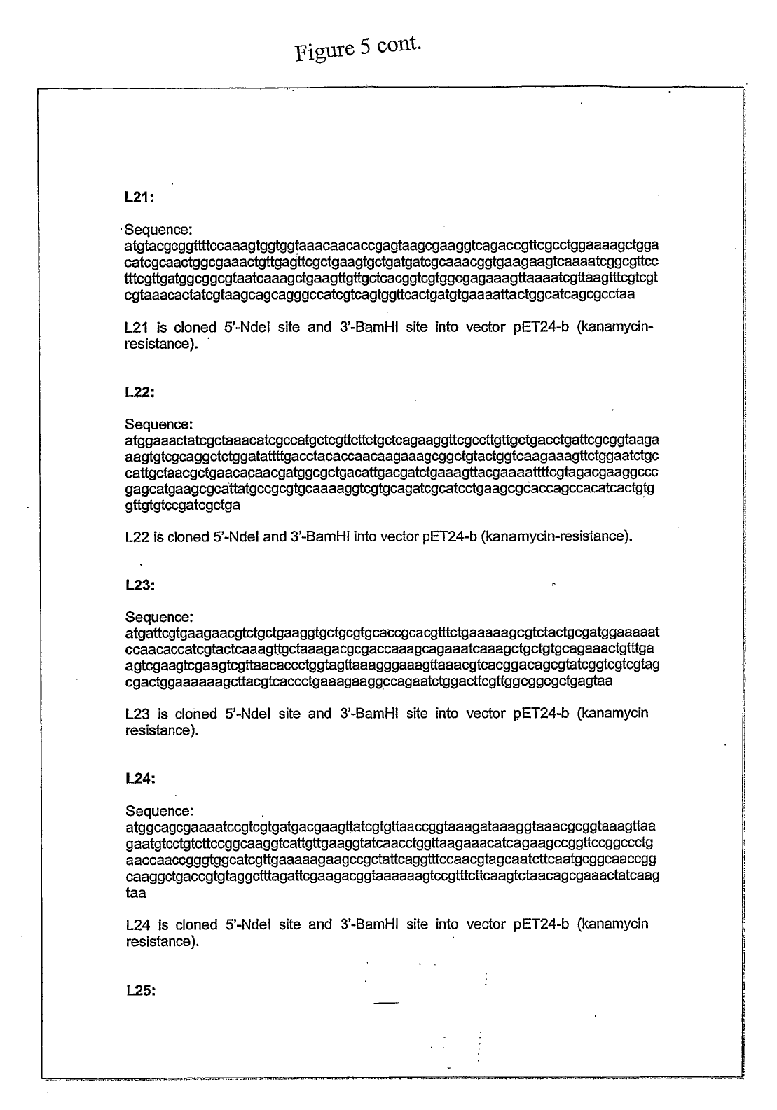

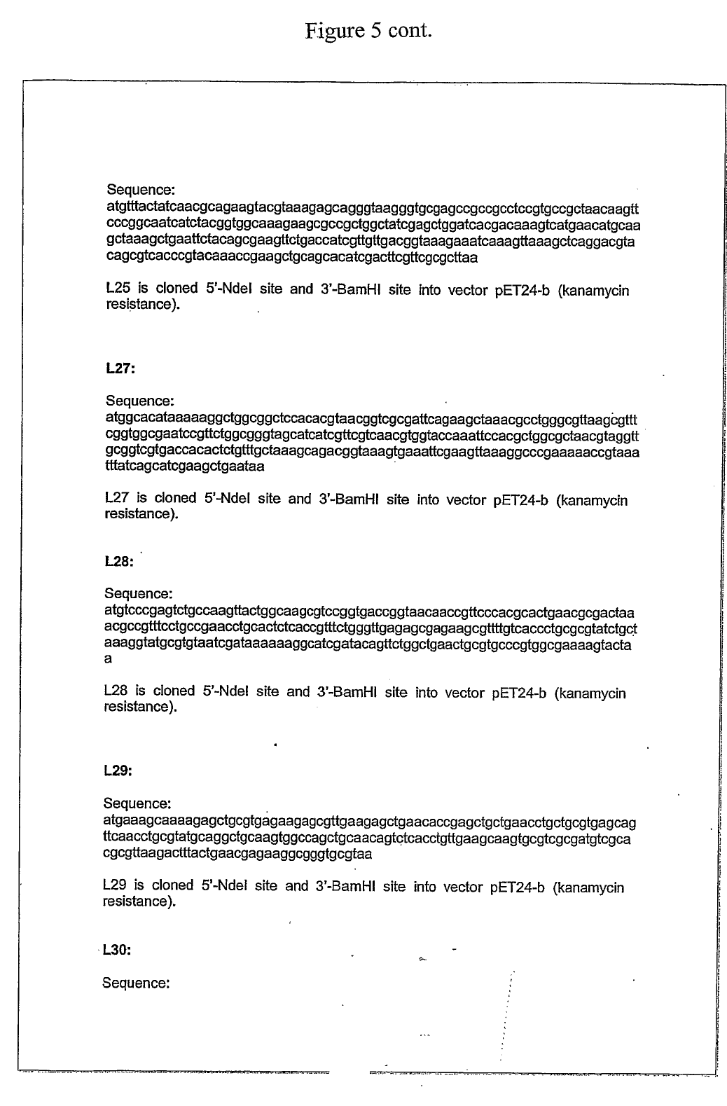

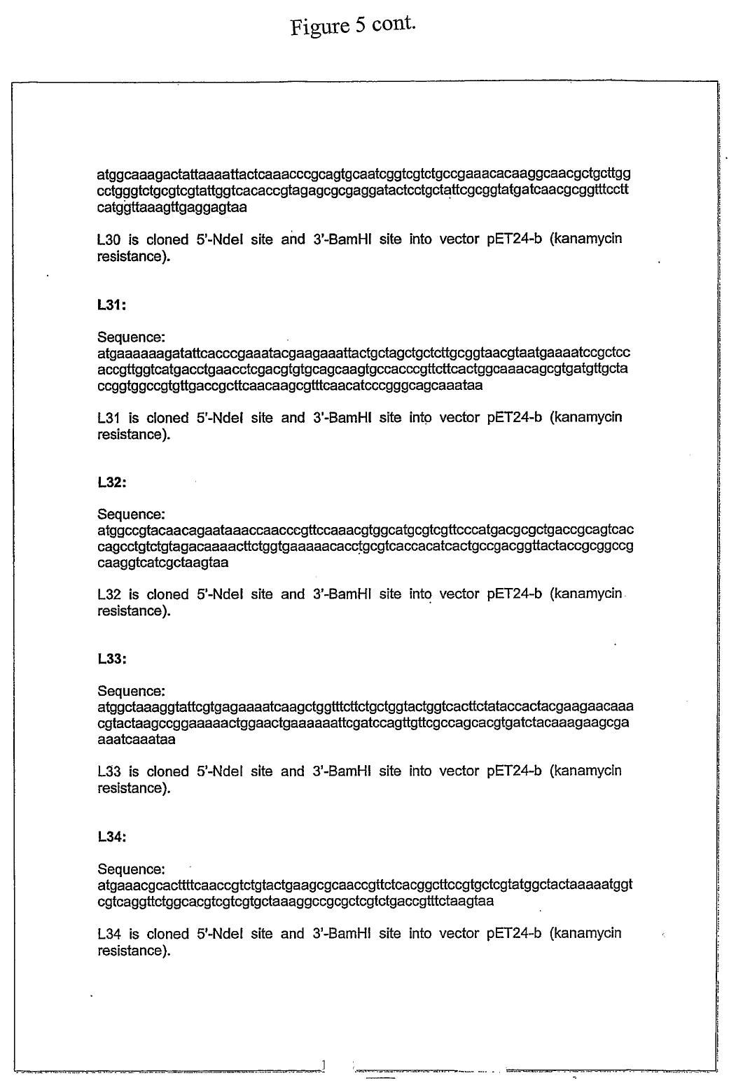

As described in more detail above, the standard reconstitution protocol for E. coli 50S subunits involves a first incubation step at low temperature (44° C.) and low magnesium (4 mM), which is then followed by incubation at high temperature (50° C.) and high magnesium (20 mM; Dohme & Nierhaus, 1976). A schematic diagram showing these steps is provided at FIG. 4. To investigate at which step a reagent affects the assembly process, reagents are added at various points during the reconstitution protocol. In one embodiment, a candidate compound is added to the reconstitution reaction at the start of the reconstitution so that the compounds are present throughout the entire protocol. In another embodiment, the compounds are added at the start of reconstitution but dialyzed after the first step of reconstitution at 44° C. In another embodiment, both compounds are added after the first and before the second step of incubation. In yet another embodiment, both compounds are added at the completion of the incubations to samples sitting at 4° C.

The reconstitution reactions are dialyzed to remove the stimulatory agents prior to assaying the peptidyl transferase activity. As controls, the compounds are assayed to assess their effect on reconstitution with a natural transcript or on peptidyl transferase activity with natural 50S subunits. In one preferred embodiment, telithromycin and TMAO are present during the entire reconstitution procedure.

The reconstitution of functional ribonucleoprotein complexes can be optimized using standard procedures to enhance subunit function. In one embodiment, compounds that increase the overall efficiency of the in vitro reconstitution reaction are selected. Briefly, a standard reconstitution reaction containing in vitro-transcribed 23S rRNA, 5S rRNA, and recombinant TP50 are incubated with reagents (e.g., antibiotics, RNA chaperones, chemical chaperones, and denaturants) at varying concentrations. If desired, the added reagent is removed from the reconstitution reaction by dialysis overnight prior to assessing the reconstitution efficiency. Methods for assaying subunit function include the Fragment Assay and the Intact tRNA Assay.

### Fragment Assay for Peptidyl Transferase Activity

The activity of a ribonucleoprotein complex is assessed using any suitable method, including, for example, either of two different peptidyl transferase assays delineated herein.

Fragment Assay

The fragment reaction, which was described by Green & Noller, 1996 (supra) utilizes minimal tRNA substrates (CAACCA-N-Ac-Methionine and puromycin) to follow the formation of a single peptide bond on isolated 50S subunits, independent of the 30S subunit or added mRNA template (Monro & Marcker, J Mol Biol. Apr. 28, 1967;25(2):347-50). The products of the reactions are resolved by paper electrophoresis and quantitated as previously described (Kim & Green, Mol Cell. November;4(5):859-64. 1999). Overall, the fragment reaction is expected to be the most sensitive to irregularities in the reconstituted particles due to the low binding affinity of the minimal substrates.

Intact tRNA Assay

A second assay uses intact aminoacylated tRNA (N-Ac-Phe-tRNAPhe) as a peptidyl tRNA substrate and radioactively labeled [³²P]-CPm (cytidyl-puromycin) as a minimal aminoacyl substrate. The intact tRNA assay is performed essentially as described (Thompson et al., Proc Natl Acad Sci USA. 98(16):9002-7, 2001). In brief, these substrates are incubated with recombinant 30S subunits programmed with poly-uridine mRNA and a recombinant 50S subunit. The formation of the dipeptidyl product is monitored by polyacrylamide gel electrophoresis (Kim & Green, supra). In the intact tRNA assay, binding interactions with the natural 30S subunit by the anticodon end of the P-site substrate can compensate for some of the binding deficiencies of a reconstituted 50S particle.

### Ribonucleoprotein Complexes Having Altered Substrate Specificity

Ribonucleoprotein complexes that accept altered substrates are described, for example, by Noren et al., (Science 244:182-188, 1989) and Bain et al, (Tetrahedron 47:2389-2400, 1991); also known in the art are ribonucleoprotein and multimolecular complexes capable of synthesizing translation products having altered backbones (See Fahnestock et al, Science 173: 340-343; Gooch et al, Biochem J. 149:209-220); and methods of producing nonstandard polymers or “drug-like” methylated peptides via ribosome-based encoding and in vitro selection, as described by Merryman and Green, Chemistry & Biology 11:575-582, 2004.

### Methods for Identifying Ribonucleoprotein Complexes Having Altered Substrate Capability

Methods of selecting ribonucleoprotein complexes having altered substrate capability or capable of synthesizing biopolymers are known in the art and are described, for example, by Cochella and Green (Proc Natl Acad Sci USA. Mar. 16, 2004;101(11):3786-91); and by Dedkova et al., (J Am Chem Soc. Jun. 4, 2003;125(22):6616-7); and in U.S. Pat. Nos. 6,358,713 by Green et al., and in U.S. Patent Publication Nos. 20040038273, 20030022213, and 20020031762. Ribonucleoprotein complexes and multimolecular complexes capable of accepting unnatural substrates and synthesizing biopolymers are particularly useful in the methods of the invention.

### An In Vitro System for Studying rRNA Mutants

Mutant rRNAs (e.g., E. coli, B. stearothermophilus and T. aquaticus) (Polacek et al., RNA. 7(10):1365-9. 2001; Thompson et al., Proc Natl Acad Sci USA. 98(16):9002-7, 2001)) are transcribed and incorporated by reconstitution into ribonucleoprotein subunits and multimolecular complexes. The effects of mutations at particular sites within the complexes is then assessed using a peptidyl transferase assays. In one embodiment, mutations are made in a site that plays a role in the catalysis of peptide bond formation (Ban et al., Science 289(5481):905-20 2000). The activity of complexes containing mutations is compared to reconstituted complexes not containing mutations or is compared to wild type particles. Methods for introducing mutations into a component of a ribonucleoprotein complex include site-directed mutagenesis and in vitro transcription. The presence of mutations is confirmed by sequencing.

### Reconstituted 30S Subunits

Reconstituted ribonucleoprotein complexes having the function of naturally occurring 30S ribosomal particles are known in the art. (See Culver and Noller, Methods Enzymol. 318:446-60, 2000; Grondek et al., RNA 10:1861-1866; Holmes et al., Nature Structural & Molecular Biology 11:179-186; Culver 68:234-249, 2003).

### Recombinant Multimolecular Ribonucleoprotein Complexes

The methods of the invention provide for the assembly of a multimolecular complex containing at least two ribonucleoprotein complexes, where one complex has the biological activity of a naturally occurring 30S subunit and one has the biological activity of a naturally occurring 50S subunit. Such a multimolecular complex is the functional equivalent of an artificial ribosome. The multimolecular complex provides for the in vitro synthesis of proteins and biopolymers. Such complexes can be assembled using, for example, in vitro transcribed prokaryotic ribosomal RNAs (e.g., 5S, 23S, 16S) and recombinant prokaryotic ribosomal proteins (e.g., L1-L34, S1-S21).

In general, a reconstituted 30S ribosomal subunit generated according to methods known in the art is combined with a 50S ribosomal subunit generated as described herein, under conditions that allow for the assembly of a multimolecular complex. In brief, such multimolecular ribonucleoprotein complexes typically form when two independently synthesized 30S and 50S subunits are combined. Reconstituted subunits generated by ordered assembly or co-transcriptional assembly are both useful for the generation of a 70S multimolecular complex. Alternatively, 5S and 23S rRNA is transcribed in vitro in the presence of a mixture of recombinant L1-L34; optionally, this in vitro transcription reaction is carried out in combination with an in vitro transcription reaction for a 30S subunit, where a 16S rRNA is in vitro transcribed in the presence of S1-S21 proteins. In other embodiments, the 30S reaction occurs before, during, or after the 50S co-transcriptional assembly reaction.

The presence of a template is not required for multimolecular complex formation. In association with a template encoding a protein or biopolymer, the multimolecular complex is capable of in vitro protein or biopolymer synthesis. The biological activity of the multimolecular complex, which is functionally equivalent to a 70S prokaryotic ribosome, is assayed by measuring, for example, poly-phe synthesis,the rate of in vitro protein synthesis, or by assaying the biological activities of the component ribonucleoprotein complex subunits, which are functionally equivalent to 30S and 50S ribosomal subunits.

Poly-Phe Synthesis

The biological activity of a multimolecular complex (e.g., an in vitro assembled 70S ribosome particle) is assessed using any translation assay known in the art. (See, for example, Southworth et al., J Mol Biol. 324(4):611-23, 2002). In one embodiment, a 70S particle is supplied with a poly-uridine mRNA template and pre-charged Phe-tRNA-Phe, elongation factors EF-Tu, EF-Ts and EF-G, and GTP and the efficiency of poly-phe synthesis is evaluated using acid precipitation (TCA) to determine the amount of polypeptide generated.

In vitro Translation

An in vitro translation reaction can be used to generate proteins or biopolymers. Methods for in vitro translation are described, for example, by Merryman et al., (Chemistry & Biology, Vol. 9, 741-746, June, 2002). In one embodiment, a natural mRNA template, such as the gene 32 mRNA template from bacteriophage T4, is used for in vitro translation. An isolated multimolecular complex is incubated with reagents containing aminoacyl tRNA synthetases, initiation factors, elongation factors, and release factors, in combination with a tRNA mixture, an amino acid mixture, and appropriate amounts of energy. The synthesis of protein products corresponding to an mRNA template can be used to evaluate the efficiency of the subunit reconstitution reaction, or to generate in vitro synthesized proteins. Such methods are also useful for the in vitro generation of biopolymers.

Methods for enhancing the assembly of the multimolecular complex and for modulating the translation rate, substrate acceptance, and translational fidelity are known in the art and are also described herein.

### Prokaryotic Ribonucleoprotein Complexes

The skilled artisan appreciates that while the Examples describe the isolation and characterization of E. coli L1-L34 proteins, which are components of the E. coli 50S subunit, the invention is not so limited. Also within the scope of the invention are “orthologs” of ribosomal proteins L1-L34. An ortholog is a protein or nucleic acid molecule of an organism that is highly related to a reference protein or nucleic acid sequence from another organism. The degree of relatedness may be expressed as the probability that a reference protein would identify a sequence, for example, in a BLAST search. The probability that a reference sequence would identify a random sequence as an ortholog is extremely low, less than e⁻¹⁰, e⁻²⁰, e⁻³⁰, e⁻⁴⁰, e⁻⁵⁰, e⁻⁷⁵, or e⁻¹⁰⁰. The skilled artisan understands that an ortholog is likely to be functionally related to the reference protein or nucleic acid sequence. In other words, the ortholog and its reference molecule would be expected to fulfill similar, if not equivalent, functional roles in their respective organisms. In one embodiment, an ortholog of a ribosomal proteins is able to substitute for the reference protein in a functional assay of biological activity.

It is not required that an ortholog, when aligned with a reference sequence, have a particular degree of amino acid sequence identity to the reference sequence. A protein ortholog might share significant amino acid sequence identity over the entire length of the protein, for example, or, alternatively, might share significant amino acid sequence identity over only a single functionally important domain of the protein. Such functionally important domains may be defined by genetic mutations or by structure-function assays. Orthologs may be identified using methods provided herein. The functional role of an ortholog may be assayed using methods well known to the skilled artisan, and described herein. For example, function might be assayed in vivo or in vitro using a biochemical, immunological, or enzymatic assay; transformation rescue, or other assay described herein. Preferred assays for ribosomal protein activity include assays for RNA chaperone activity, protein chaperone activity, RNA binding activity, or protein binding activity. In other embodiments, function is assessed in a cell in culture; function may also be assayed by gene inactivation (e.g., by RNAi, siRNA, or gene knockout), or gene over-expression, as well as by other methods.

Virtually any prokaryotic ribosomal nucleic acid molecule, protein, or analog thereof is useful in the methods of the invention. In particular embodiments, the prokaryote is a bacteria or archaea. Of particular interest are prokaryotes that are mesophiles, thermophiles, moderate thermophiles, extreme thermophiles, hyperthermophiles, and psychrophile.

Exemplary bacteria useful in the methods of the invention include Actinobacteria, Aquificae, Bacteroidetes/Chlorobi group, Chlamydiae/Verrucomicrobia group, Chloroflexi, Chrysiogenetes, Cyanobacteria, Deferribacteres, Deinococcus-Thermus, Dictyoglomi, Fibrobacteres/Acidobacteria group, Firmicutes, Fusobacteria, Gemmatimonadetes, Nitrospirae, Planctomycetes, Proteobacteria, Spirochaetes, Thermodesulfobacteria, and Thermotogae. In particular, exemplary bacteria useful in the methods of the invention include, but are not limited to, Aerobacter, Aeromonas, Acinetobacter, Actinomyces israelli, Agrobacterium, Bacillus, Bacillus antracis, Bacteroides, Bartonella, Bordetella, Bortella, Borrelia, Brucella, Burkholderia, Calymmatobacterium, Campylobacter, Citrobacter, Clostridium, Clostridium perfringers, Clostridium tetani, Cornyebacterium, corynebacterium diphtheriae, corynebacterium sp., Enterobacter, Enterobacter aerogenes, Enterococcus, Erysipelothrix rhusiopathiae, Escherichia, Francisella, Fusobacterium nucleatum, Gardnerella, Haemophilus, Hafnia, Helicobacter, Klebsiella, Klebsiella pneumoniae, Legionella, Leptospira, Listeria, Morganella, Moraxella, Mycobacterium, Neisseria, Pasteurella, Pasturella multocida, Proteus, Providencia, Pseudomonas, Rickettsia, Salmonella, Serratia, Shigella, Staphylococcus, Stentorophomonas, Streptococcus, Streptobacillus moniliformis, Treponema, Treponema pallidium, Treponema pertenue, Xanthiomonas, Vibrio, and Yersinia.

Exemplary archaea include Crenarchaeota, Euryarchaeota, Korarchaeota, Nanoarchaeota and unclassified Archaea. In particular, archaea useful in the methods of the invention include, but are not limited to, Archaeoglobi, Halobacteria, Methanobacteria, Methanococci, Methanomicrobia, Methanopyri, Thermococci, Thermoplasmata, Aeropyrum pernix, Archaeoglobus fulgidus, Halobacterium sp., Methanococcus jannaschii, Methanobacterium thermoautotrophicum, Pyrococcus abyssi, Pyrococcus horikoshii, and Thermoplasma acidophilum

Prokaryotic ribosomes are known in the art and are described, for example, in U.S. Pat. Nos. 6,638,908, which describes the crystal structure of the large ribosomal subunit of Haloarcula maris (Protein Database Accession No.: 1FFZ and 1FG0). Other U.S. Patents and Patent Publications that describe prokaryotic ribosomes include 20030232779, 20030171327, and 20020086308 by Steitz et al.; and 20020188108 by Noller et al. The sequences of exemplary prokaryotic ribosomal proteins are also described in the following EMBL Accession Nos. Ribosomal protein L1: Genename: rplA, (rpy); EMBL Acc. No.: EMBL;V00339; ECRPOBC (12337). EMBL;U00006; ECUW89 (176195) EMBL;AE000472; ECAE472 (14659) Swiss Prot. Acc No: P02384; RL1_ECOLI; Ribosomal protein L2: Genename: rplB; EMBL Acc. No.: EMBL;X02613; ECRPOS10 (5422). EMBL;U18997; ECUW67 (372438); EMBL;AE000408; ECAE408 (10944); Swiss Prot. Acc No: P02387; RL2_ECOLI; Ribosomal protein L3: Genename: rplC; EMBL Acc. No.: EMBL;V00344; ECRPSL (1241); EMBL;U18997; ECUW67 (372438); EMBL;X02613; ECRPOS10 (5422); EMBL;AE000408; ECAE408 (10944); Swiss Prot. Acc No: P02386; RL3_ECOLI; Ribosomal protein L4: Genename: rplD; EMBL Acc. No.: EMBL;X02613; ECRPOS10 (5422). EMBL;U18997; ECUW67 (372438); EMBL;AE000408; ECAE408 (10944). Swiss Prot. Acc No: P02388; RL4_ECOLI; Ribosomal protein L5: Genename: rplE; EMBL Acc. No.: EMBL;X01563; ECSPC (5922). EMBL;U18997; ECUW67 (372438). EMBL;M10195; EMBL;AE000408; ECAE408 (10944).

Swiss Prot. Acc No: P02389; RL5_ECOLI; Ribosomal protein L6: Genename: rplF; EMBL Acc. No.: EMBL;X01563; ECSPC (5922). EMBL;U18997; ECUW67 (372438).EMBL;AE000408; ECAE408 (10944); Swiss Prot. Acc No: P02390; RL6_ECOLI; Ribosomal protein L7/12: Genename: rplL; EMBL Acc. No.: EMBL;M38332; EMBL;V00339; ECRPOBC (12337). EMBL;U00006; ECUW89 (176195).EMBL;AE000472; ECAE472 (14659); Swiss Prot. Acc No: P02392; RL7_ECOLI; Ribosomal protein L9: Genename: rplI; EMBL Acc. No.: EMBL;X04022; ECRPSFRI (1979). EMBL;U14003; EC14003 (338534); EMBL;AE000491; ECAE491 (11152); Swiss Prot. Acc No: P02418; RL9_ECOLI; Ribosomal protein L10: Genename: rplJ; EMBL Acc. No.: EMBL;V00339; ECRPOBC (12337). EMBL;U00006; ECUW89 (176195); EMBL;AE000472; ECAE472 (14659); Swiss Prot. Acc No: P02408; RL10_ECOLI; Ribosomal protein L11: Genename: rplK; EMBL Acc. No.: EMBL;V00339; ECRPOBC (12337). EMBL;U00006; ECUW89 (176195); EMBL;M30610; ECSECE (1380). EMBL;AE000472; ECAE472 (14659); Swiss Prot. Acc No: P02409; RL11_ECOLI; Ribosomal protein L13: Genename: rplM; EMBL Acc. No.: EMBL;X02130; ECRPSI (1184). EMBL;U18997; ECUW67 (372438); EMBL;AE000402; ECAE402 (10713); Swiss Prot. Acc No: P02410; RL13_ECOLI; Ribosomal protein L14: Genename: rplN; EMBL Acc. No.: EMBL;X01563; ECSPC (5922). EMBL;U18997; ECUW67 (372438). EMBL;V00357; EMBL;X02613; ECRPOS10 (5422). EMBL;AE000408; ECAE408 (10944); Swiss Prot. Acc No: P02411; RL14_ECOLI; Ribosomal protein L15: Genename: rplO; EMBL Acc. No.: EMBL;X01563; ECSPC (5922). EMBL;U18997; ECUW67 (372438); EMBL;AE000408; ECAE408 (10944); Swiss Prot. Ace No: P02413; RL15_ECOLI; Ribosomal protein L16: Genename: rplP; EMBL Acc. No.: EMBL;M12490; EMBL;X02613; ECRPOS10 (5422). EMBL;U18997; ECUW67 (372438). EMBL;AE000408; ECAE408 (10944); Swiss Prot. Acc No: P02414; RL16_ECOLI; Ribosomal protein L17: Genename: rplQ; EMBL Acc. No.: EMBL;X00766; ECRPOA (1424). EMBL;U18997; ECUW67 (372438); EMBL;L29458; ECYHDM (1592). EMBL;X02543; ECRPA (3154). EMBL;AE000407; ECAE407 (10602). EMBL;X53844; ECRPOA15 (987); Swiss Prot. Acc No: P02416; RL17_ECOLI; Ribosomal protein L18: Genename: rplR; EMBL Acc. No.: EMBL;X01563; ECSPC (5922). EMBL;U18997; ECUW67 (372438); EMBL;AE000408; ECAE408 (10944); Swiss Prot. Acc No: P02419; RL18_ECOLI; Ribosomal protein L19: Genename: rplS EMBL Acc. No.: EMBL;X01818; ECTRMD (4586). EMBL;AE000346; ECAE346 (12129); Swiss Prot. Acc No: P02420; RL19_ECOLI: Ribosomal protein L20: Genename: rplT (pdzA); EMBL Acc. No.: EMBL;K02844; ECHIMA (5972). EMBL;V00291; ECTHRINF (7784). EMBL;M10423; ECHIMB (500). EMBL;D90813; ECD813 (19273). EMBL;D90814; ECD814 (19031); EMBL;AE000266; ECAE266 (10558); Swiss Prot. Acc No: P02421; RL20_ECOLI. Ribosomal protein L21: Genename: rplU; EMBL Acc. No.: EMBL;D13267; ECRPLRPM (2529). EMBL;U18997; ECUW67 (372438); EMBL;AE000399; ECAE399 (17800); Swiss Prot. Acc No: P02422; RL21_ECOLI. Ribosomal protein L22: Genename: rplV; EMBL Acc. No.: EMBL;X02613; ECRPOS10 (5422). EMBL;U18997; ECUW67 (372438); EMBL;AE000408; ECAE408 (10944); Swiss Prot. Acc No: P02423; RL22_ECOLI; Ribosomal protein L23: Genename: rplW; EMBL Acc. No.: EMBL;X02613; ECRPOS10 (5422). EMBL;U18997; ECUW67 (372438); EMBL;AE000408; ECAE408 (10944); Swiss Prot. Acc No: P02424; RL23_ECOLI; Ribosomal protein L24: Genename: rplX; EMBL Acc. No.: EMBL;M10195; EMBL;X01563; ECSPC (5922). EMBL;U18997; ECUW67 (372438); EMBL;AE000408; ECAE408 (10944); Swiss Prot. Acc No: P02425; RL24_ECOLI; Ribosomal protein L25: Genename: rplY; EMBL Acc. No.: EMBL;D13326; ECD13326 (1116). EMBL;U00008; ECHU49 (39149); EMBL;AE000308; ECAE308 (11961); Swiss Prot. Acc No: P02426; RL25_ECOLI; Ribosomal protein L27: Genename: rpmA; EMBL Acc. No.: EMBL;D13267; ECRPLRPM (2529). EMBL;U18997; ECUW67 (372438); EMBL;AE000399; ECAE399 (17800); Swiss Prot. Acc No: P02427; RL27_ECOLI; Ribosomal protein L28: Genename: rpmB; EMBL Acc. No.: EMBL;L10328; ECUW82 (136254). EMBL;J01677; ECRPMBG (764); EMBL;AE000441; ECAE441 (10562); Swiss Prot. Acc No: P02428; RL28_ECOLI; Ribosomal protein L29: Genename: rpmC; EMBL Acc. No.: EMBL;X02613; ECRPOS10 (5422). EMBL;U18997; ECUW67 (372438); EMBL;AE000408; ECAE408 (10944); Swiss Prot. Acc No: P02429; RL29_ECOLI.

Ribosomal protein L30: Genename: rpmD; EMBL Acc. No.: EMBL;X01563; ECSPC (5922). EMBL;U18997; ECUW67 (372438). EMBL;AE000408; ECAE408 (10944); Swiss Prot. Acc No: P02430; RL30_ECOLI; Ribosomal protein L31: Genename: rpmE; EMBL Acc. No.: EMBL;M21516; ECKATGA (2805). EMBL;L19201; ECUW87 (96484); EMBL;D00616; ECPRIAY (2907). EMBL;AE000467; ECAE467 (15633); Swiss Prot. Acc No: P02432; RL31_ECOLI; Ribosomal protein L32: Genename: rpmF; EMBL Acc. No.: EMBL;M29698; ECRPMFA (1191). EMBL;D90744; ECD744 (14663); EMBL;AE000209; ECAE209 (10438). EMBL;M96793; ECPLSFABA (1428); Swiss Prot. Acc No: P02435; RL32_ECOLI; Ribosomal protein L33: Genename: rpmG; EMBL Acc. No.: EMBL;J01677; ECRPMBG (764). EMBL;L10328; ECUW82 (136254); EMBL;X06036; ECFPG (1093). EMBL;AE000441; ECAE441 (10562); Swiss Prot. Acc No: P02436; RL33_ECOLI; Ribosomal protein L34: Genename: rpmH, (rinA,ssaF;); EMBL Acc. No.: EMBL;L10328; ECUW82 (136254). EMBL;J01602; ECDNAAN (3873); EMBL;M11056; ECRNPA (1069). EMBL;AE000447; ECAE447 (11354); Swiss Prot. Acc No: P02437; RL34_ECOLI.

Polynucleotides encoding prokaryotic ribosomal proteins are described in the following publications:
Blattner et al., 1997. The complete genome sequence of Escherichia coli K-12. Science 277:1453-1462;
Bult et al., 1996. Complete genome sequence of the methanogenic archeon, Methanococcus jannaschii. Science 273:1058-1073;
Cole et al. 1998. Deciphering the biology of Mycobacterium tuberculosis from the complete genome sequence. Nature 393:537-544.
Condo et al., 1999. Cis-acting signals controlling translational initiation in the thermophilic archaeon Sulfolobus solfataricus. Mol. Microbiol. 34:377-384.
Deckert et al., 1998. The complete genome of the hyperthermophilic bacterium Aquifex aeolicus. Nature 392:353-358.
Fleischmann et al. 1995. Whole-genome random sequencing and assembly of Haemophilus influenzae Rd. Science 269:496-512;
Fraser et al. 1997. Genomic sequence of a Lyme disease spirochete, Borrelia burgdorferi. Nature 390:580-586;
Fraser et al. 1995. The minimal gene complement of Mycoplasma genitalium. Science 270:397-403;
Fraser et al. 1998. Complete genome sequence of Treponema pallidum, the syphilis spirochete. Science 281:375-388;
Ge et al., 1999. Contributions of genome sequencing to understanding the biology of Helicobacter pylori. Annu. Rev. Microbiol. 53:353-387;
Himmelreich et al., 1996. Complete sequence analysis of the genome of the bacterium Mycoplasma pneumoniae. Nucleic Acids Res. 24:4420-4449;
Haft et al. 1999. Evidence for lateral gene transfer between Archaea and Bacteria VOL. 182, 2000 PHX GENES OF DIVERSE PROKARYOTIC GENOMES 5249;
Kalman et al., 1999. Comparative genomes of Chlamydia pneumoniae and C. trachomatis. Nat. Genet. 21:385-389;
Kaneko et al., 1996. Sequence analysis of the genome of the unicellular cyanobacterium Synechocystis sp. PCC6803. II. Sequence determination of the entire genome and assignment of potential protein-coding regions. DNA Res. 3:109-136;
Saito, R., and M. Tomita. 1999. Computer analyses of complete genomessuggest that some archaebacteria employ both eukaryotic and eubacterial mechanisms in translation initiation. Gene 238:79-83;
Kawarabayasi et al., 1998. Complete sequence and gene organization of the genome of a hyper-thermophilic archaebacterium, Pyrococcus horikoshii OT3. DNARes. 5:55-76;
Kikuchi. 1998. Complete sequence and gene organization of the genome of a hyper-thermophilic archaebacterium, Pyrococcus horikoshii OT3. DNA Res. 5:55-76;
Klenk et al. 1997. The complete genome sequence of the hyperthermophilic, sulphate-reducing archaeon Archaeoglobus fulgidus. Nature 390:364-370;
Kunst et al. 1997. The completegenome sequence of the Gram-positive bacterium Bacillus subtilis. Nature 390:249-256
Stephens et al., 1998. Genome sequence of an obligate intracellular pathogen of humans: Chlamydia trachomatis. Science 282:754-759;
Tomb et al., 1999. Computer analyses of complete genomes suggest that some archaebacteria employ both eukaryotic and eubacterial mechanisms in translation initiation. Gene 238:79-83;
Smith et al. 1997. Complete genome sequence of Methanobacterium thermoautotrophicum delta H: functional analysis and comparative genomics. J. Bacteriol. 179:7135-7155; and
Stephens et al., 1998. Genome sequence of an obligate intracellular pathogen of humans: Chlamydia trachomatis. Science 282:754-759.

### Protein Expression

In general, ribosomal proteins (e.g., L1-L34) of the invention may be produced by transformation of a suitable host cell with all or part of a protein-encoding nucleic acid molecule or fragment thereof in a suitable expression vehicle.

Those skilled in the field of molecular biology will understand that any of a wide variety of expression systems may be used to provide the recombinant protein. The precise host cell used is not critical to the invention. A protein of the invention may be produced in a prokaryotic host (e.g., E. coli) or in a eukaryotic host (e.g., Saccharomyces cerevisiae, insect cells, e.g., SF21 cells, or mammalian cells, e.g., NIH 3T3, HeLa, or preferably COS cells). Such cells are available from a wide range of sources (e.g., the American Type Culture Collection, Rockland, Md.; also, see, e.g., Ausubel et al., supra). The method of transformation or transfection and the choice of expression vehicle will depend on the host system selected. Transformation and transfection methods are described, e.g., in Ausubel et al. (supra); expression vehicles may be chosen from those provided, e.g., in Cloning Vectors: A Laboratory Manual (P. H. Pouwels et al., 1985, Supp. 1987).

A variety of expression systems exist for the production of the proteins of the invention. Expression vectors useful for producing such proteins include, without limitation, chromosomal, episomal, and virus-derived vectors, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof.

One particular bacterial expression system for protein production is the E. coli pET expression system (Novagen, Inc., Madison, Wis.). According to this expression system, DNA encoding a protein is inserted into a pET vector in an orientation designed to allow expression. Since the gene encoding such a protein is under the control of the T7 regulatory signals, expression of the protein is achieved by inducing the expression of T7 RNA polymerase in the host cell. This is typically achieved using host strains that express T7 RNA polymerase in response to IPTG induction. Once produced, recombinant protein is then isolated according to standard methods known in the art, for example, those described herein.

Another bacterial expression system for protein production is the pGEX expression system (Pharmacia). This system employs a GST gene fusion system that is designed for high-level expression of genes or gene fragments as fusion proteins with rapid purification and recovery of functional gene products. The protein of interest is fused to the carboxyl terminus of the glutathione S-transferase protein from Schistosoma japonicum and is readily purified from bacterial lysates by affinity chromatography using Glutathione Sepharose 4B. Fusion proteins can be recovered under mild conditions by elution with glutathione. Cleavage of the glutathione S-transferase domain from the fusion protein is facilitated by the presence of recognition sites for site-specific proteases upstream of this domain. For example, proteins expressed in pGEX-2T plasmids may be cleaved with thrombin; those expressed in pGEX-3X may be cleaved with factor Xa.

Once the recombinant protein of the invention is expressed, it is isolated, e.g., using affinity chromatography. In one example, an antibody (e.g., produced as described herein) raised against a protein of the invention may be attached to a column and used to isolate the recombinant protein. Lysis and fractionation of protein-harboring cells prior to affinity chromatography may be performed by standard methods (see, e.g., Ausubel et al., supra).

Once isolated, the recombinant protein can, if desired, be further purified, e.g., by high performance liquid chromatography (see, e.g., Fisher, Laboratory Techniques In Biochemistry and Molecular Biology, eds., Work and Burdon, Elsevier, 1980). Proteins of the invention, particularly short peptide fragments, can also be produced by chemical synthesis (e.g., by the methods described in Solid Phase Peptide Synthesis, 2nd ed., 1984 The Pierce Chemical Co., Rockford, Ill.). These general techniques of protein expression and purification can also be used to produce and isolate useful peptide fragments or analogs (described herein).

### Ribosomal Protein Analogs

Also included in the invention are ribosomal proteins (e.g., L1-L34) or fragments thereof that are modified or altered in ways that enhance or do not inhibit their ability to function in a ribosomal subunit. In one embodiment, the invention provides methods for optimizing a ribosomal protein (e.g., L1-L34) amino acid sequence or nucleic acid sequence by producing an alteration. Such changes may include certain mutations, deletions, insertions, or post-translational modifications. The invention further includes analogs of any naturally-occurring protein of the invention. Analogs can differ from the naturally-occurring protein of the invention by amino acid sequence differences, by post-translational modifications, or by both. Analogs of the invention will generally exhibit at least 85%, more preferably 90%, and most preferably 95% or even 99% identity with all or part of a naturally-occurring amino, acid sequence of the invention. The length of sequence comparison is at least 10, 13, 15 amino acid residues, preferably at least 25 amino acid residues, and more preferably more than 35 amino acid residues. Again, in an exemplary approach to determining the degree of identity, a BLAST program may be used, with a probability score between e⁻³ and e⁻¹⁰⁰ indicating a closely related sequence.

Modifications include in vivo and in vitro chemical derivatization of proteins, e.g., acetylation, carboxylation, phosphorylation, or glycosylation; such modifications may occur during protein synthesis or processing or following treatment with isolated modifying enzymes. Analogs can also differ from the naturally-occurring proteins of the invention by alterations in primary sequence. These include genetic variants, both natural and induced (for example, resulting from random mutagenesis by irradiation or exposure to ethanemethylsulfate or by site-specific mutagenesis as described in Sambrook, Fritsch and Maniatis, Molecular Cloning: A Laboratory Manual (2d ed.), CSH Press, 1989, or Ausubel et al., supra). Also included are cyclized peptides, molecules, and analogs which contain residues other than L-amino acids, e.g., D-amino acids or non-naturally occurring or synthetic amino acids, e.g., beta, or gamma amino acids.

“Conservatively modified variants” apply to both amino acid and nucleic acid sequences. With respect to particular nucleic acid sequences, conservatively modified variants refers to those nucleic acids which encode identical or similar amino acid sequences and include degenerate sequences. For example, the codons GCA, GCC, GCG and GCU all encode alanine. Thus, at every amino acid position where an alanine is specified, any of these codons can be used interchangeably in constructing a corresponding nucleotide sequence. The resulting nucleic acid variants are conservatively modified variants, since they encode the same protein (assuming that is the only alternation in the sequence). One skilled in the art recognizes that each codon in a nucleic acid, except for AUG (sole codon for methionine) and UGG (tryptophan), can be modified conservatively to yield a functionally-identical peptide or protein molecule.

As to amino acid sequences, one skilled in the art will recognize that substitutions, deletions, or additions to a protein or protein sequence which alter, add or delete a single amino acid or a small number (typically less than about ten) of amino acids is a “conservatively modified variant” where the alteration results in the substitution of an amino acid with a chemically similar amino acid. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparigine or glutamine; isoleucine to leucine or valine; leucine to valine or isoleucine; lysine to arginine, glutamine, or glutamate; methionine to leucine or isoleucine; phenylalanine to tyrosine, leucine or methionine; serine to threonine; threonine to serine; tryptophan to tyrosine; tyrosine to tryptophan or phenylalanine; valine to isoleucine or leucine. Other conservative and semi-conservative substitutions are known in the art and can be employed in practice of the present invention.

In addition to full-length proteins, the invention also includes fragments of any one of the proteins of the invention. As used herein, the term “a fragment” means at least 5, 10, 15, or 20 amino acids. In other embodiments a fragment is at least 50, 100, 150, or 200 contiguous amino acids, and in other embodiments at least 250, 300, 400, 500 or more contiguous amino acids. Fragments of the invention can be generated by methods known to those skilled in the art or may result from normal protein processing (e.g., removal of amino acids from the nascent protein that are not required for biological activity or removal of amino acids by alternative mRNA splicing or alternative protein processing events).

Non-protein ribosomal protein (e.g., L1-L34) analogs having a chemical structure designed to mimic ribosomal protein (e.g., L1-L34) functional activity can be administered according to methods of the invention. Ribosomal protein (e.g., L1-L34) analogs may exceed the physiological activity of native ribosomal proteins (e.g., L1-L34). Methods of analog design are well known in the art, and synthesis of analogs can be carried out according to such methods by modifying the chemical structures such that the resultant analogs exhibit the biological activity of a native ribosomal protein. These chemical modifications include, but are not limited to, substituting alternative R groups and varying the degree of saturation at specific carbon atoms of the native Ribosomal protein (e.g., L1-L34) molecule. Assays for measuring functional activity include, but are not limited to, those described in the Examples below.

### Microarrays

The ribosomal proteins and nucleic acid molecules of the invention, or fragments thereof, are useful as hybridizable array elements in a microarray. The nucleic acid molecules or proteins may be arranged in a grid where the location of each nucleic acid molecule or protein remains fixed to aid in identification of the individual nucleic acid molecules or proteins. A microarray may include, for example, nucleic acid molecules representing all, or a subset, of the open reading frames of an organism, or of the proteins that those open reading frames encode. A microarray may also be enriched for a particular type of gene. In one example, a microarray of “ribosomal nucleic acid molecules or proteins” may be enriched for nucleic acid molecules or their encoded proteins so that, for example, it comprises at least 5%, 10%, 15%, 20%, 22%, 25%, 30%, 40%, 50%, 60%, 70%, 80%, 90%, 95%, 97% or even 99% nucleic acid molecule required for ribosomal subunit assembly.

The array elements are typically organized in an ordered fashion such that each element is present at a specified location on the substrate. Useful substrate materials include membranes, composed of paper, nylon or other materials, filters, chips, glass slides, and other solid supports. The ordered arrangement of the array elements allows hybridization patterns and intensities to be interpreted as expression levels of particular genes or proteins. Methods for making nucleic acid microarrays are known to the skilled artisan and are described, for example, in U.S. Pat. No. 5,837,832, Lockhart, et al. (Nat. Biotech. 14:1675-1680, 1996), and Schena, et al. (Proc. Natl. Acad. Sci. 93:10614-10619, 1996), herein incorporated by reference. Methods for making protein microarrays are described, for example, by Ge (Nucleic Acids Res. 28: e3. i-e3. vii, 2000), MacBeath et al., (Science 289:1760-1763, 2000), Zhu et al.(Nature Genet. 26:283-289), and in U.S. Pat. No. 6,436,665, hereby incorporated by reference.

Nucleic Acid Microarrays

To produce a nucleic acid microarray oligonucleotides may be synthesized or bound to the surface of a substrate using a chemical coupling procedure and an ink jet application apparatus, as described in PCT application W095/251116 (Baldeschweiler et al.), incorporated herein by reference. Alternatively, a gridded array may be used to arrange and link cDNA fragments or oligonucleotides to the surface of a substrate using a vacuum system, thermal, UV, mechanical or chemical bonding procedure.

A nucleic acid molecule (e.g., RNA or DNA) derived from a biological sample may be used to produce a hybridization probe as described herein. The biological samples are generally derived from a patient, preferably as a bodily fluid (such as blood, cerebrospinal fluid, phlegm, saliva, or urine) or tissue sample (e.g., a tissue sample obtained by biopsy). For some applications, cultured cells (e.g., lymphocytes) or other tissue preparations may be used. The mRNA is isolated according to standard methods, and cDNA is produced and used as a template to make complementary RNA suitable for hybridization. Such methods are described herein. The RNA is amplified in the presence of fluorescent nucleotides, and the labeled probes are then incubated with the microarray to allow the probe sequence to hybridize to complementary oligonucleotides bound to the microarray.

Incubation conditions are adjusted such that hybridization occurs with precise complementary matches or with various degrees of less complementarity depending on the degree of stringency employed. For example, stringent salt concentration will ordinarily be less than about 750 mM NaCl and 75 mM trisodium citrate, preferably less than about 500 mM NaCl and 50 mM trisodium citrate, and most preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and most preferably at least about 50% formiamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37 in 500 mM NaCl, 50 mM trisodium citrate, 1% SDS, 35% formamide, and 100 μg/ml denatured salmon sperm DNA (ssDNA). In a most preferred embodiment, hybridization will occur at 42 in 250 mM NaCl, 25 mM trisodium citrate, 1% SDS, 50% formanide, and 200 μg/ml ssDNA. Useful variations on these conditions will be readily apparent to those skilled in the art.

The removal of nonhybridized probes may be accomplished, for example, by washing. The washing steps that follow hybridization can also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and most preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a most preferred embodiment, wash steps will occur at 68° C. in 15 mM NaCl, 1.5 mM trisodium citrate, and 0.1% SDS. Additional variations on these conditions will be readily apparent to those skilled in the art.

A detection system may be used to measure the absence, presence, and amount of hybridization for all of the distinct sequences simultaneously (e.g., Heller et al., Proc. Natl. Acad. Sci. 94:2150-2155, 1997). Preferably, a scanner is used to determine the levels and patterns of fluorescence.

Protein Microarrays

Ribosomal proteins (e.g., L1-L34) may also be analyzed using protein microarrays. Such arrays are useful in high-throughput low-cost screens to identify peptide or candidate compounds that bind a protein of the invention, or fragment thereof. Typically, protein microarrays feature a protein (e.g., a ribosomal protein), or fragment thereof, bound to a solid support. Suitable solid supports include membranes (e.g., membranes composed of nitrocellulose, paper, or other material), polymer-based films (e.g., polystyrene), beads, or glass slides. For some applications, proteins (e.g., proteins encoded by a nucleic acid molecule or antibodies against such proteins) are spotted on a substrate using any convenient method known to the skilled artisan (e.g., by hand or by inkjet printer). Preferably, such methods retain the biological activity or function of the protein bound to the substrate (Ge et al., supra; Zhu et al., supra).

The protein microarray is hybridized with a detectable probe. Such probes can be protein, nucleic acid, or small molecules. For some applications, protein and nucleic acid probes are derived from a biological sample taken from a patient, such as a bodily fluid (such as blood, urine, saliva, or phlegm); a homogenized tissue sample (e.g. a tissue sample obtained by biopsy); or cultured cells (e.g., lymphocytes). Probes can also include antibodies, candidate peptides, nucleic acids, or small molecule compounds derived from a peptide, nucleic acid, or chemical library. Hybridization conditions (e.g., temperature, pH, protein concentration, and ionic strength) are optimized to promote specific interactions. Such conditions are known to the skilled artisan art are described, for example, in Harlow, E. and Lane, D., Using Antibodies : A Laboratory Manual. 1998, New York: Cold Spring Harbor Laboratories. After removal of non-specific probes, specifically bound probes are detected, for example, by fluorescence, enzyme activity (e.g., an enzyme-linked calorimetric assay), direct immunoassay, radiometric assay, or any other suitable detectable method known to the skilled artisan.

### Screening Assays

As discussed above, a ribosomal protein (e.g., L1-L34) or fragment thereof is useful in reconstituting ribosomal activity in vitro. Accordingly, compounds that enhance the activity of a ribosomal protein (e.g., L1-L34) or fragment thereof are useful in the methods of the invention. Any number of methods are available for carrying out screening assays to identify such compounds. In one approach, candidate compounds are identified that specifically bind to and enhance the activity of a protein of the invention. Of particular interest are compounds that enhance the activity of a reconstituted ribosome (e.g., any biological activity that enhances protein synthesis, rRNA binding, tRNA binding, peptidyl transferase activity, mRNA translocation activity, or ribosomal assembly). Methods of assaying such activities are known in the and are described herein. The efficacy of such a candidate compound is dependent upon its ability to interact with a ribosomal protein (e.g., L1-L34) or nucleic acid molecule. Such an interaction can be readily assayed using any number of standard binding techniques and functional assays (e.g., those described in Ausubel et al., supra). For example, a candidate compound may be tested in vitro for interaction and binding with a protein or nucleic acid molecule of the invention and its ability to enhance the activity of a reconstituted ribosome may be assayed by any standard assays (e.g., those described herein).

Potential agonists include organic molecules, peptides, peptide mimetics, proteins, nucleic acid ligands, and antibodies that bind to a nucleic acid sequence or protein of the invention and thereby inhibit or extinguish its activity. Potential antagonists also include small molecules that bind to and occupy the binding site of the protein thereby preventing binding to cellular binding molecules, such that normal biological activity is prevented.

In one particular example, a candidate compound that binds to a ribosomal protein (e.g., L1-L34) or fragment thereof maybe identified using a chromatography-based technique. For example, a recombinant ribosomal protein of the invention may be purified by standard techniques from cells engineered to express the protein (e.g., those described above) and may be immobilized on a column. A solution of candidate compounds is then passed through the column, and a compound specific for the ribosomal protein (e.g., L1-L34) is identified on the basis of its ability to bind to the ribosomal protein (e.g., L1-L34) and be immobilized on the column. To isolate the compound, the column is washed to remove non-specifically bound molecules, and the compound of interest is then released from the column and collected. Compounds isolated by this method (or any other appropriate method) may, if desired, be further purified (e.g., by high performance liquid chromatography). Compounds isolated by this approach may also be used, for example, as therapeutics to treat or prevent the onset of a pathogenic infection, disease, or both. Compounds that are identified as binding to ribosomal proteins (e.g., L1-L34) or fragment thereof with an affinity constant less than or equal to 10 mM are considered particularly useful in the invention.

### Test Compounds and Extracts

In general, compounds capable of enhancing the activity of a ribosomal protein (e.g., L1-L34) or fragment thereof are identified from large libraries of either natural product or synthetic (or semi-synthetic) extracts or chemical libraries according to methods known in the art. Those skilled in the field of drug discovery and development will understand that the precise source of test extracts or compounds is not critical to the screening procedure(s) of the invention. Accordingly, virtually any number of chemical extracts or compounds can be screened using the methods described herein. Examples of such extracts or compounds include, but are not limited to, plant-, fingal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Furthermore, if desired, any library or compound is readily modified using standard chemical, physical, or biochemical methods.

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their anti-pathogenic activity should be employed whenever possible.

When a crude extract is found to enhance the biological activity of a ribosomal protein (e.g., L1-L34), GCRR, or fragment thereof, further fractionation of the positive lead extract is necessary to isolate chemical constituents responsible for the observed effect. Thus, the goal of the extraction, fractionation, and purification process is the careful characterization and identification of a chemical entity within the crude extract having anti-pathogenic activity. Methods of fractionation and purification of such heterogenous extracts are known in the art. If desired, compounds shown to be useful agents for enhancing the activity of a reconstituted ribosome are chemically modified according to methods known in the art.

### EXAMPLES

The eubacterial ribosome is a complex macromolecular machine composed of a small (30S) subunit including 16S rRNA and more than 20 proteins and a large (50S) subunit consisting of 23S and 5S rRNAs and more than 30 proteins. Many of the ribosomal proteins have unusual extended basic tails that appear to wind their way through ribosomal RNA, acting to cement together the final structure. Most ribosomal proteins also have a globular domain that extends over the surface of the subunit, most often in solvent exposed regions. There are, however, notable exceptions where the ribosomal proteins are located in the interface region between the large and small subunits of the ribosome where they might play direct roles in functionally critical intersubunit interactions like S12 and S13 (Cukras et al., 2003; Culver et al., 1999). In addition, while ribosomal proteins are largely absent from the peptidyl transfer and decoding region, they are the principal components of the stalk region of the large ribosomal subunit that is directly involved in binding and interacting with translation factors like EF-Tu and EF-G. Finally, a variety of ribosomal proteins are directly implicated in ribosome function based on documentation of their motions during discrete steps of translation (Gao et al., 2003; Valle et al., 2003).

Earlier work from Belfort and co-workers demonstrated that the small subunit ribosomal protein S12 from E. coli has ATP-independent RNA annealing and RNA displacement activities in vitro (Coetzee et al., Genes Dev 8:1575-1588,1994). These activities are associated with RNA chaperones because their actions increase the fraction of correctly folded RNA by either impeding the formation of misfolded structures or by destabilizing folding intermediates (Herschlag, 1995). In a separate work the relevance of the in vitro activity of ribosomal protein S12 could be extended to an in vivo context where S12 overexpression rescued a misfolded group I intron to promote catalysis (Clodi et al., EMBO J 18:3776-3782, 1999). In vivo it was further demonstrated that the RNA chaperone StpA destabilizes tertiary structural elements of a group I intron, again ultimately leading to correct folding and associated catalysis (Waldsich et al., Genes Dev. 16:2300-2312, 2002). While it is unclear if the documented RNA chaperone activity of S12 is implemented by the ribosome for optimal function, it is clear that such activities might be useful to guarantee the dynamic flexibility of the ribosome during the translation cycle.

To extend the initial observation made with S12, the purification of the ribosomal proteins from the large ribosomal subunit from E. coli and their evaluation in an in vitro RNA chaperone assay was carried out according to methods described by Coetzee et al. Nearly a third of the E. coli large ribosomal subunit proteins assayed displayed RNA chaperone activity.

### Example 1

### A Third of the E. coli Large Ribosomal Subunit Proteins Have RNA Chaperone Activity

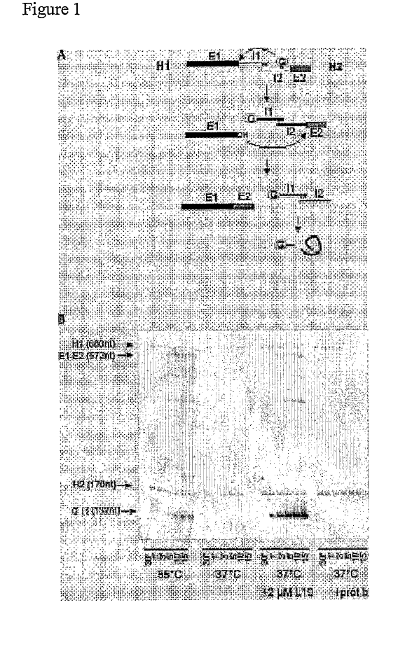

The ribosomal proteins were cloned, over-expressed and purified using methods previously described (Culver and Noller, RNA 5:832-843, 1999). The previously developed trans splicing assay was used to evaluate the RNA chaperone activity of the collection of purified ribosomal proteins (Coetzee et al., 1994, supra). In this assay the pre-mRNA of the thymidylate synthase (td) gene containing a group I intron is split in two halves. The first transcript, H1, contains the 5′ exon sequences and 95 nucleotides of the intron and the second transcript, H2, contains the 3′ part of the intron and parts of exon 2 (FIG. 1A).

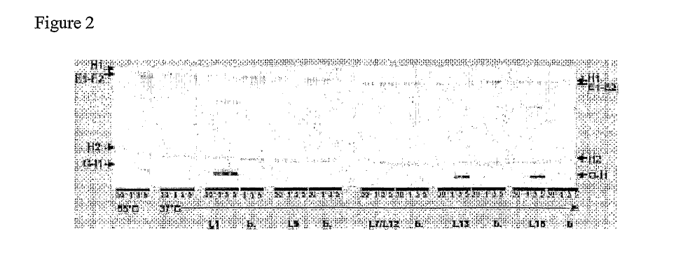

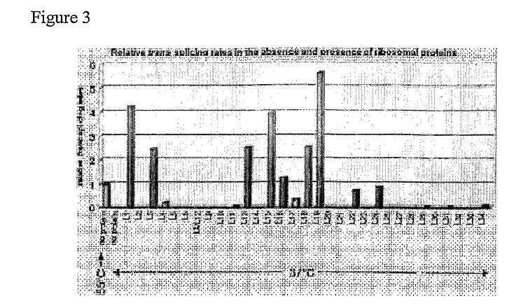

The two independent constructs were transcribed using ³⁵S-UTP and were then incubated together. The group I intron trans-splicing reaction was initiated with the addition of ³²P-GTP, so that the resulting spliced product is doubly labeled (internally with ³⁵S-UTP and at the 5′ end with GTP). Trans splicing was performed in the absence and the presence of the respective ribosomal proteins. At 55° C. the HI and H2 RNAs effectively annealed and folded into a productive molecule while at 37° C. the RNAs failed to assemble into an active conformation. The group I splicing reaction is effectively cold sensitive. The individual purified ribosomal proteins were added to a final concentration of 2 μM as was used recently for assessing the RNA chaperone activity of StpA (Mayer et al., Biochem Soc Trans 30:1175-1180, 2002). Higher protein concentrations (3 μM, 4 μM, 6 μM) were tested for ribosomal protein L19 and did not show an increase in the observed chaperoning activity. Addition of an RNA chaperone at 55° C. does not further stimulate trans splicing. FIG. 1B shows a representative time course of trans splicing in the presence of ribosomal protein L19 where the reaction is 5-times more efficient with L19 at 37° C. than without L19 at 55° C. (positive control). Ribosomal proteins L1, L13, and L15 among others, further increased trans splicing at 37° C., whereas others (L9, L7/L12 did not stimulate trans splicing (FIG. 2). FIG. 3 shows the relative trans splicing rates of proteins from the large ribosomal subunit. Proteins L1, L3, L13, L15, L16, L18, L19, L22 and L24 significantly stimulated trans splicing. More moderate stimulation of trans splicing was observed with L4 and L17.

### Example 2

### Combining Ribosomal Proteins L4 and L24 Did Not Further Increase Trans Splicing Rate

Next, a combination of two proteins was assayed to determine whether the combination further increased the splicing rates. Ribosomal proteins L4 and L24 both associate with a short fragment within 23S rRNA (Stelzl and Nierhaus, RNA 7:598-609, 2001), suggesting that they might act cooperatively. Addition of L4 and L24 did not further stimulate trans splicing.

### Example 3

### Ribosomal Proteins L1 and L19 Have High Levels of RNA Chaperone Activity

Ribosomal proteins L1 and L19 showed the highest levels of RNA chaperone activity in our assay. (L1: relative splicing rate 4-times over positive control, L19: relative splicing rate 5-times over positive control). Ribosomal protein L19 is located at the subunit interface and interacts with L14, L3 and with rRNA elements of both subunits (Harms et al., Cell 107:679-688, 2001). Ribosomal protein L1 consists of two globular domains and is localized to the stalk region near the E-site. Previous studies indicate that this is a highly flexible region of the ribosome (Stark et al., Cell 100:301-309, 2000; Uchiumi et al., J. Biol. Chem. 277:41401-41409, 2002). It has been further suggested that L1 plays a role in facilitating E-site tRNA release, an activity that may be facilitated by RNA chaperone activity (Agrawal et al., Int. J. Biochem Cell Biol, 31:243-254, 1999) (Gomez-Lorenzo et al., EMBO J 19:2710-2718, 2000) (Yusupov et al., Science 292:883-896, 2001) (Harms et al., Cell 107:679-688, 2001). Finally, L1 is a translational repressor that regulates expression of its own mRNA in E. coli (Nomura et al., Ann Rev Biochem 53:75-117, 1984) (Nikonov et al., EMBO J., 15:1350-1359, 1996). While general models for translation control by several ribosomal proteins are based on sequestration of the Shine-Dalgarno region, it is possible that these proteins directly resolve RNA structures to promote their own repressor activities. A similar model has recently been proposed for another RNA chaperone, Hfq, which binds to and resolves RNA structural elements (Geissmann and Touati, EMBO J. 23:396-405, 2004). Ribosomal protein L3 is one of the early assembling proteins (Rohl and Nierhaus, PNAS 79:729-733, 1982) and is composed of a globular domain and an unstructured extension (Ban et al., supra; Harms et al., supra). The unstructured extension has a methylated glutamine residue and the loss of this modification results in cold sensitive ribosome assembly (Chang and Chang, Biochem. 14-468-477, 1975) (Lhoest and Colson, Eur. J. Biochem., 121:33-37, 1981). Perhaps L3 directly facilitates RNA rearrangements in this region that are critical in preventing the long-term sampling of cold sensitive metastable states. Finally, like L1, ribosomal protein L4 is a negative transcriptional and translational regulator of its own operon (Lindahl and Zengel, PNAS 76:6542-6546, 1979) (Yates et al., PNAS 77:1837-1841, 1980; Zengel et al., 1980), again consisting of a globular domain with a long unstructured extension, like L3 (Ban et al., supra; Harms et al., supra). Both L4 and L22 are located along the exit tunnel of the ribosome suggesting potential roles in regulating the extrusion of the growing peptide (Nissen et al., Science 289:920-930, 2000) (Gabashvili et al., Mol Cell 8:181-188, 2001).

Most of the ribosomal proteins are located at the periphery of the ribosome and are thought to play critical roles both in assembly and stabilization of the ribosome structure. Some of the ribosomal proteins that have significant RNA chaperone activity in our assay assemble at early stages of reconstitution, (e.g.: L3 and L4) (Nierhaus, Biochimie 73:739-755, 1991). Other identified proteins are involved at later stages during assembly (e.g.: L15, L16) (Franceschi and Nierhaus, J. Biol. Chem. 265:16676-16682, 1990). It is likely that RNA chaperone activity functions at all stages of assembly, ensuring that the RNA does not become trapped in non-native structures. It is also possible that the ribosomal proteins play critical roles in facilitating RNA rearrangements during translation, and that these movements depend on the RNA chaperone-like properties of these unusual proteins. Without being tied to one particular theory, it is possible that the RNA chaperone activity of the ribosomal proteins identified here, besides preventing the formation of misfolded structures during assembly, could be more generally critical to the dynamic rearrangements of the ribosome during tRNA selection, peptide bond formation and translocation.

The experiments described above were carried out using the following methods.

### Cloning, Expression and Purification of Ribosomal Proteins:

Genomic DNA from E. coli MRE600 was used for PCR amplification. The PCR primers were chosen to have NdeI (5′) and BamHI (3′) sites except for ribosomal protein L16 where primers with Ndel and EcoRI sites were used. The PCR product was cloned into the vector pET24b (Novagen). The ribosomal proteins were over expressed in E. coli strain BLR1. After 4 hours of induction the cells were collected, resuspended in 20 mM Tris pH 7.0 at 4° C., 1 M KCI, 6 mM β mercaptoEtOH and cracked with French Press. The lysate was centrifuged by 4° C. to either clear the supernatant or pellet the insoluble protein. The pellet was solubilized in the respective buffer. Resuspended pellet or supernatant were dialysed at 4° C. over night into the respective FPLC buffer (Table 1). The lysate was then purified over a resource S cation column (or a resource Q anion column).

TABLE 1

| Ribosomal | | FPLC | | Elution | | |
|---|---|---|---|---|---|---|
| Protein | Solubility | column^{a} | Buffer^{b} | (mM KC1) | p1^{c} | Mw^{d} |
| L1 | insoluble | Resource S | A, B | 270, 210 | 9.2 | 24.599 |
| L2 | insoluble | Resource S | B, B | 280 | >12.0 | 29.730 |
| L3 | insoluble | Resource S | C, C | 175 | 9.7 | 22.258 |
| L4 | insoluble | Resource Q | C, D | 180, 55 | 7.6 | 22.087 |
| L5 | insoluble | Resource S | B, A | flowthrough | 9.4 | 20.171 |
| L6 | purified by Julie Brunelle | (unpublished) | | | 10.0 | 18.832 |
| L7/L12 | purified by Julie Brunelle | (unpublished) | | | 4.8 | ~12.2 each |
| L9 | insoluble | Resource S | B, C | flowthrough | 6.4 | 15.696 |
| LI0 | purified by Julie Brunelle | (unpublished) | | | 7.5 | 17.581 |
| L11 | purified by Julie Brunelle | (unpublished) | | | 9.7 | 14.874 |
| L13 | insoluble | Resource S | A, B | 255, 215 | 10.1 | 16.019 |
| L 14 | purified by Julie Brunelle | (unpublished) | | | 12.3 | 13.341 |
| L15 | soluble | Resource S | B | 260 | 12.0 | 14.981 |
| L16 | insoluble | Resource S | B, B | 320 | 12.0 | 15.296 |
| L17 | soluble | Resource S | B, D | 205 | 11.0 | 14.365 |
| L18 | insoluble | Resource S | B, D | 280, 215 | 12.0 | 12.770 |
| L19 | insoluble | ResourceS | B, B | 180 | >12.0 | 13.002 |
| L20 | soluble | Resource S | B, B | 360 | >12.0 | 13.366 |
| L21 | insoluble | Resource S | C | 800 | 8.2 | 11.565 |
| L22 | soluble | Resource S | A, A | 160 | 11.5 | 12.227 |
| L23 | soluble | Resource S | C, A | 535, 200 | 9.6 | 12.209 |
| L24 | soluble | Resource S | A, A | 155 | 10.7 | 11.185 |
| L25 | insoluble | Resource S | C | 345 | 9.4 | 10.694 |
| L27 | soluble | Resource S | B, B | 220 | >12.0 | 8993 |
| L28 | insoluble | Resource S | B, A | 320, 365 | 11.42 | 8875 |
| L29 | insoluble | Resource S | A, B | 90, 60 | 10.0 | 7274 |
| L30 | soluble | Resource S | B, D | 200 | >12.0 | 6411 |
| L31 | soluble | Resource S | A, B | 230, 160 | 9.46 | 6971 |
| L32 | soluble | Resource S | B, B | 210 | 11.3 | 6315 |
| L33 | insoluble | Resource S | B, B | 225 | >12.0 | 6255 |
| L34 | soluble | Resource S | B | 385 | >12.0 | 5381 |

Table 1:
Resource S: cation column;
resource Q: anion column;
^{a}Buffer used for dialysis and for the FPLC column. A: 20 mM Tris (pH 7.0 at 4° C.), 6 M urea, 20 mM KCI B: 20 mM Tris (pH 8.0 at 4° C.), 6 M urea, 20 mM KCI C: 20 mM NaAc (pH 5.6), 6 M urea, 20 mM KCI D: 20 mM Tris (pH 9.5 at 4° C.), 6 M urea, 20 mM KCI Ribosomal proteins were purified up to 2 times each depending on their purity.
^{b}pI from (Kaltschmidt, 1971), pIs printed in italic were calculated from http://us.expasy.org/tools/pi_tool.html
^{c}Molecular weights from (Giri et al., 1984)

The proteins were eluted over a salt gradient (20 mM KCl-1000 mM KICl) and dialyzed into storage buffer (20 mM Tris pH 7.4, 4 mM MgAc₂, 400 mM NH₄Cl, 0.2 mM EDTA, 5 mM (β-mercaptoEtOH) except for L4, which was stored in buffer C (Table 1) and dialysed into storage buffer prior to usage. The purity of the ribosomal proteins was tested on Coomassie stained SDS urea gels.

The ribosomal proteins were further tested for RNase activity by incubating the ribosomal protein with transcribed RNA and testing RNA degradation on a polyacrylamide (PAA) gel. Each ribosomal protein was purified until no significant RNase activity was left.

### In vitro Transcription

The plasmids for H1 (exon 1+5′intron) and H2 (3′ intron+exon 2) (Coetzee et al., 1994) were linearized with SapI (for H1) and BamH1 (for H2). The RNAs H1 and H2 were transcribed with 40 mM Tris pH 7.0, 26 mM MgCl₂, 3 mM spermidine, 5 mM ATP, 5 mM GTP, 5 mM CTP, 2.5 mM UTP, 2.5 mM ³⁵S-α-UTP, 10 mM DTT, T7 RNA polymerase at 37° C. for 3 hours, followed by a thirty minute DNase digest and purification of the transcripts over 5% PAA gel electrophoresis.

### Trans Splicing Assay

200 nM H1 and 200 nM 112 transcripts were incubated for 1 minute at 95° C. and cooled to either 55° C. (for the positive control) or 37° C. Next, splicing buffer (4 mM Tris pH 7.4, 3 mM MgCl₂, 0.4 mM spermidine, 4 mM DTT final concentration) and 0.33 pMol ³²P-GTP were added. Then, either the respective ribosomal protein was added to a final concentration of 2 μM or the same quantity of ribosomal storage buffer (20 mM Tris pH 7.4, 4 mM MgAc₂, 400 mM NH₄CL, 0.2 mM EDTA, 5 mM (β-mercaptoEtOH) was added. The reactions were incubated at either 55° C. (positive control) or at 37° C. and aliquots were stopped by adding a final concentration of 40 mM EDTA and 300 μg/ml tRNA. The samples were phenol-CHCl₃ extracted, precipitated and loaded on 5% PAA gels. Bands were measured by PhosphoImager and relative rates were calculated for each gel by setting the positive control to 1 and subtracting the negative control (trans splicing at 37° C. in the absence of ribosomal proteins) from each obtained rate on each gel by the formula: (nx-n37)/(n55-n37), nx being the relative splicing rate in the presence of the respective ribosomal protein, n55 being the relative splicing rate at 55° C. in the absence of ribosomal proteins and n37 being the relative splicing rate at 37° C. in the absence of a ribosomal protein.

### Other Embodiments

From the foregoing description, it will be apparent that variations and modifications may be made to the invention described herein to adopt it to various usages and conditions. Such embodiments are also within the scope of the following claims. All patents and publications mentioned in this specification are herein incorporated by reference to the same extent as if each independent patent and publication was specifically and individually indicated to be incorporated by reference.

1. An isolated ribonucleoprotein complex comprising a recombinant prokaryotic ribosomal polynucleotide and a recombinant prokaryotic ribosomal protein, or fragments thereof, wherein the ribonucleoprotein complex has a biological activity of a reference prokaryotic 50S ribosomal subunit.

2. An isolated ribonucleoprotein complex comprising a recombinant polynucleotide having at least 85% nucleic acid sequence identity to a reference prokaryotic ribosomal polynucleotide sequence and a recombinant protein having at least 85% amino acid sequence identity to a reference prokaryotic ribosomal protein sequence, or fragments thereof, wherein the ribonucleoprotein complex has a biological activity of a naturally occurring prokaryotic 50S ribosomal subunit.

3. An isolated ribonucleoprotein complex comprising a recombinant ribosomal prokaryotic polynucleotide of a bacteria and a recombinant ribosomal protein of a bacteria, or fragments thereof, wherein the ribonucleoprotein complex has a biological activity of a reference bacterial 50S ribosomal subunit.

4. The ribonucleoprotein complex of claim 3, wherein the prokaryote is a bacterium selected from the group consisting of Actinobacteria, Aquificae, Bacteroidetes/Chlorobi group, Chlamydiae/Vetrucomicrobia group, Chloroflexi, Chrysiogenetes, Cyanobacteria, Deferribacteres, Deinococcus-Thermus, Dictyoglomi, Fibrobacteres/Acidobacteria group, Firmicutes, Fusobacteria, Gemmatimonadetes, Nitrospirae, Planctomycetes, Proteobacteria, Spirochaetes, Thermodesulfobacteria, Tliermotogae and unclassified bacteria.

5. The ribonucleoprotein complex of claim 4, wherein the bacterium is selected from the group consisting of Aerobacter, Aeromonas, Acinetobacter, Actinomyces israelii, Agrobacterium, Bacillus, Bacillus antracis, Bacteroides, Bartonella, Bordetella, Bortella, Borrelia, Brucella, Burkholderia, Calymmatobacterium, Campylobacter, Citrobacter, Clostridium, Clostridium perfringers, Clostridium tetani, Corny ebacterium, corynebacterium diphtheriae, corynebacterium sp., Enter obacter, Enterobacter aerogenes, Enterococcus, Erysipelothrix rhusiopathiae, Escherichia, Francisella, Fusobacterium nucleatum, Gardnerella, Haemophilus, Hafnia, Helicobacter, Klebsiella, Klebsiella pneumoniae, Legionella, Leptospira, Listeria, Morganella, Moraxella, Mycobacterium, Neisseria, Pasteurella, Pasturella multocida, Proteus, Providencia, Pseudomonas, Rickettsia, Salmonella, Serratia, Shigella, Staphylococcus, Stentorophomonas, Streptococcus, Streptobacillus moniliformis, Treponema, Treponema pallidium, Treponema pertenue, Xanthomonas, Vibrio, and Yersinia.

6. The ribonucleoprotein complex of claim 5, wherein the recombinant protein and polynucleotide is at least 85% identical to a ribosomal protein or polynucleotide of Escherichia coli, and the ribonucleoprotein complex has a biological activity of a naturally occurring Escherichia coli 50S ribosomal subunit.

7. The ribonucleoprotein complex of claim 6, wherein the recombinant protein and polynucleotide is at least 90% identical to a ribosomal protein or polynucleotide of Escherichia coli, and the ribonucleoprotein complex has a biological activity of a naturally occurring Escherichia coli 50S ribosomal subunit.

8. The ribonucleoprotein complex of claim 7, wherein the recombinant protein and polynucleotide is at least 95% identical to a ribosomal protein or polynucleotide of Escherichia coli, and the ribonucleoprotein complex has a biological activity of a naturally occurring Escherichia coli 50S ribosomal subunit.

9. An isolated ribonucleoprotein complex comprising a recombinant ribosomal polynucleotide of Archaea and a recombinant ribosomal protein of Archaea, or fragments thereof, wherein the ribonucleoprotein complex has a biological activity of a reference 50S ribosomal subunit of Archaea.

10. The ribonucleoprotein complex of claim 9, wherein the prokaryote is an archaea selected from the group consisting of Crenarchaeota, Euryarchaeota, Korarchaeota, Nanoarchaeota and unclassified Archaea.

11. The ribonucleoprotein complex of claim 10, wherein the Crenarchaeota is a Thermoprotei.

12. The ribonucleoprotein complex of claim 10, wherein the Euryarchaeota is selected from the group consisting of Archaeoglobi, Halobacteria, Methanobacteria, Methanococci, Methanomicrobia, Methanopyri, Thermococci, and Tltermoplasmata.

13. The ribonucleoprotein complex of claim 9, wherein the Archaea is selected from the group consisting of Aeropyrum pernix, Archaeoglobus fulgidus, Halobacterium sp., Methanococcus jannaschii, Methanobacterium thermoautotrophicum, Pyrococcus abyssi, Pyrococcus horikoshii, and Thermoplasma acidophilum.

14. The ribonucleoprotein complex of claim 1, wherein the biological activity is peptidyl transferase activity.

15. The ribonucleoprotein complex of claim 1, wherein the biological activity is a protein-protein or protein-RNA interaction that can be detected by mass spectroscopy.

16. The ribonucleoprotein complex of claim 1, wherein the prokaryote is selected from the group consisting of a mesophile.

17. The ribonucleoprotein complex of claim 1, wherein the prokaryote is selected from the group consisting of a thermophile, a moderate thermophile, an extreme thermophile, and a hyperthermophile.

18. The ribonucleoprotein complex of claim 1, wherein the prokaryote is a psychrophile.

19. The ribonucleoprotein complex of claim 1, wherein the recombinant polynucleotide is a 5S ribosomal RNA (rRNA), or fragment thereof.

20. The ribonucleoprotein complex of claim 1, wherein the recombinant polynucleotide is a 23 S rRNA, or fragment thereof.

21. An isolated ribonucleoprotein complex comprising an in vitro transcribed prokaryotic 5 S rRNA, an in vitro transcribed 23 S rRNA, or fragments thereof, and at least one recombinant protein, selected from the group consisting of prokaryotic L1-L34 proteins, or fragments thereof, wherein the ribonucleoprotein complex has a biological activity of a reference prokaryotic 5OS ribosomal subunit.

22. An isolated ribonucleoprotein complex comprising an in vitro transcribed 5S rRNA, an in vitro transcribed 23 S rRNA, or fragments thereof, and more than one recombinant protein, or fragments thereof, selected from the group consisting of L1-L34 proteins, wherein the ribonucleoprotein complex has a biological activity of a reference 5OS ribosomal subunit.

23. An isolated ribonucleoprotein complex comprising a recombinant prokaryotic 5 S rRNA, a recombinant prokaryotic 23S rRNA, or fragments thereof, and prokaryotic ribosomal proteins L1-L34, or fragments thereof, wherein the ribonucleoprotein complex has a biological activity of a reference prokaryotic 50S ribosomal subunit.

24-34. (canceled)

35. A vector collection comprising at least two vectors, each of the vectors comprising a different polynucleotide encoding a ribosomal protein having at least 85% amino acid sequence identity to a prokaryotic ribosomal protein selected from the group consisting of L1-L34.

36-41. (canceled)

42. A primer collection, the collection comprising at least 2 sets of primers, each set capable of binding to and amplifying a different polynucleotide encoding a prokaryotic ribosomal protein selected from the group consisting of L1-L34.

43-50. (canceled)

51. A protein collection comprising at least two different recombinant proteins, each of the proteins having at least 85% amino acid sequence identity to a prokaryotic ribosomal protein selected from the group consisting of L1-L34.

52-58. (canceled)

59. A polynucleotide collection comprising at least two different polynucleotides, each of the polynucleotides encoding a protein having at least 85% amino acid sequence identity to a prokaryotic ribosomal protein selected from the group consisting of L1-L34.

60-65. (canceled)

66. A microarray comprising at least two nucleic acid molecules that encode a prokaryotic ribosomal protein selected from the group consisting of L1-L34, or fragments thereof.

67-71. (canceled)

72. A method of producing a ribonucleoprotein complex, the method comprising the steps of:
i. transcribing in vitro a prokaryotic 5S rRNA or 23S rRNA in the presence of at least one recombinant prokaryotic ribosomal protein under conditions that permit assembly of a ribonucleoprotein complex; and
ii. isolating a ribonucleoprotein complex having the biological activity of a reference 50S ribosomal subunit.

73. A method of producing an isolated ribonucleoprotein complex, the method comprising the steps of:
i. transcribing in vitro a prokaryotic 5S rRNA and a 23S rRNA in the presence of more than one recombinant prokaryotic ribosomal protein under conditions that permit assembly of a ribonucleoprotein complex; and
ii. isolating a ribonucleoprotein complex having the biological activity of a reference 50S ribosomal subunit.

74. A method of producing an isolated ribonucleoprotein complex, the method comprising:
i. transcribing in vitro a prokaryotic 5 S rRNA and a 23 S rRNA in the presence of recombinant prokaryotic ribosomal proteins L1-L34; and
ii. isolating a ribonucleoprotein complex having the biological activity of a reference 50S ribosomal subunit.

75. A method of producing an isolated ribonucleoprotein complex, the method comprising: i. contacting recombinant prokaryotic 5S rRNA and 23S rRNA with at least one recombinant prokaryotic ribosomal protein selected from the group consisting of L1-L34; and ii. isolating a ribonucleoprotein complex the biological activity of a reference 5OS ribosomal subunit.

76-100. (canceled)

101. An automated system for production of an isolated recombinant ribonucleoprotein complex, the system comprising:
i. a reaction substrate comprising reaction reagents for the production of a ribonucleoprotein complex, wherein at least one of the reaction reagents is a recombinant 5 S rRNA or a recombinant 23 S rRNA;
ii. a thermoelectric heating and cooling element on one side of the reaction substrate, wherein the element is configured to selectively heat or cool the reaction reagent;
iii. a means for selectively depositing at least one reaction reagent on the substrate, wherein the depositing means is in communication with the reaction substrate; and
iv. a means for isolating a recombinant ribonucleoprotein complex that contacts the reaction substrate.

102-103. (canceled)

104. A kit for ribonucleoprotein complex production, the kit comprising i. an in vitro transcribed prokaryotic 5S rRNA; ii. an in vitro transcribed prokaryotic 23 S rRNA; and iii. a plurality of recombinant ribosomal proteins selected from the group consisting of L1-L34.

105. A kit for ribonucleoprotein complex production, the kit comprising i. a recombinant prokaryotic 5S rRNA; ii. a recombinant prokaryotic 23S rRNA; and iii. a plurality of recombinant ribosomal proteins selected from the group consisting of L1-L34.

106. A kit for ribonucleoprotein complex production, the kit comprising i. reagents required for the in vitro transcription of a recombinant prokaryotic 5S rRNA; ii. reagents required for the in vitro transcription of a recombinant prokaryotic 23S rRNA; and iii. at least one recombinant ribosomal protein selected from the group consisting of L1-L34.

107-109. (canceled)

110. A multimolecular complex comprising: i. a 5OS ribonucleoprotein complex comprising an in vitro transcribed prokaryotic 5S rRNA, a 23 S rRNA, and at least one prokaryotic recombinant ribosomal protein selected from the group consisting of L1-L34; ii. a 30S ribonucleoprotein complex comprising an in vitro transcribed prokaryotic 16S rRNA and at least one prokaryotic recombinant ribosomal protein selected from the group consisting of S1-S21; and wherein the multimolecular complex has a biological activity of a reference 70S ribosome.

111. A multimolecular complex comprising:
i. a 50S ribonucleoprotein complex comprising a recombinant prokaryotic 5S rRNA, a recombinant 23 S rRNA, and at least one recombinant prokaryotic ribosomal protein selected from the group consisting of L1-L34; and
ii. a 30S ribonucleoprotein complex comprising a recombinant prokaryotic 16S rRNA and at least one recombinant prokaryotic ribosomal protein selected from the group consisting of S1-S21; and wherein the multimolecular complex has a biological activity of a reference 70S ribosome.

112-176. (canceled)
